(19) United States
(12) Patent Application Publication
(10) Pub. No.: US 2022/0372521 A1
(43) Pub. Date: Nov. 24, 2022

(54) RNA-GUIDED DNA INTEGRATION AND MODIFICATION

(71) Applicant: The Trustees of Columbia University in the City of New York, New York, NY (US)

(72) Inventors: Samuel Henry Sternberg, New York, NY (US); Phuc L.H. Vo, New York, NY (US)

(21) Appl. No.: 17/634,759

(22) PCT Filed: Aug. 14, 2020
(86) PCT No.: PCT/US2020/046526

§ 371 Date: Feb. 11, 2022

Related U.S. Application Data
(60) Provisional application No. 62/886,717, filed on Aug. 14, 2019.

(51) Int. Cl. C12N 15/90; C12N 15/10

(52) U.S. Cl. CPC C12N 15/90; C12N 15/102; C12N 2310/20

(57) ABSTRACT

The present disclosure provides methods and systems for resolving cointegrate products from RNA-guided DNA integration. More particularly, the present disclosure provides systems comprising: an engineered Clustered Regularly Interspaced Short Palindromic Repeats (CRISPR)-CRISPR associated (Cas) (CRISPR-Cas) system, and/or one or more vectors encoding the engineered CRISPR-Cas system, wherein the CRISPR-Cas system comprises: (a) at least one Cas protein, and (b) a guide RNA (gRNA); an engineered transposon system, and/or one or more vectors encoding the engineered transposon system, wherein the transposon system is configured for replicative transposition; a recombinase, or catalytic domain thereof, and/or one or more vectors encoding a recombinase, or catalytic domain thereof; and at least one donor nucleic acid to be integrated, wherein the donor nucleic acid comprises a recognition site for the recombinase and a cargo nucleic acid flanked by at least one transposon end sequence, and methods of use thereof.

### CROSS-REFERENCE TO RELATED APPLICATIONS

This application claims the benefit of U.S. Provisional Application No. 62/886,717, filed Aug. 14, 2019, the entire content of which is incorporated herein by reference.

### SEQUENCE LISTING STATEMENT

Incorporated by reference in its entirety herein is a computer-readable nucleotide/amino acid sequence listing submitted concurrently herewith and identified as follows: 259,817 Byte ASCII (Text) file named “ 38168-257_SEQUENCE-LISTING_ST25.TXT,” created on Jul. 20, 2022.

### FIELD

The present invention relates to methods and systems for generating and resolving cointegrate products from RNA-guided DNA integration.

### BACKGROUND

The genetic engineering toolbox for genome manipulation comprises a diverse array of techniques, with DNA insertion technologies having arguably had the largest impact on biotechnology research. Gene knock-ins are used in the clinic to treat genetic diseases and cancer, in agriculture to improve crops, and in industry to manufacture biologics, among many other uses (Dunbar, C. E. et al. Science 359, eaan4672 (2018), Gelvin, S. B. Annu Rev Genet 51, 195-217 (2017), and Wurm, F. M. Nat Biotechnol 22, 1393-1398 (2004), each incorporated herein by reference in its entirety). These applications generally depend on either site-specific integration mediated by homologous recombination and gene editing, or random integration mediated by viral integrases or transposases. The former category is inherently precise but reliant on often-inefficient cellular factors or exogenous factors with limited host range, whereas the latter category exhibits high efficiency but little specificity. For certain genome engineering challenges, the ideal technology would exhibit high-efficiency DNA integration that bypasses the requirement for DNA double-strand breaks (DSBs) and homologous recombination, but with the specificity and programmability afforded by CRISPR-Cas gene-editing platforms.

### SUMMARY

Provided herein are systems, kits, and methods that facilitate nucleic acid editing in a site-specific manner. These systems, kits, compositions, and methods employ a combination of CRISPR RNA-guided transposases and/or integrases with recombination components and other recombinase-based systems.

Provided herein are systems comprising: an engineered Clustered Regularly Interspaced Short Palindromic Repeats (CRISPR)-CRISPR associated (Cas) (CRISPR-Cas) system, and/or one or more vectors encoding the engineered CRISPR-Cas system, wherein the CRISPR-Cas system comprises: (a) at least one Cas protein, and (b) a guide RNA (gRNA); an engineered transposon system, and/or one or more vectors encoding the engineered transposon system, wherein the transposon system is configured for replicative (copy-and-paste) transposition; a recombinase, or catalytic domain thereof, and/or one or more vectors encoding a recombinase, or catalytic domain thereof; and at least one donor nucleic acid to be integrated, wherein the donor nucleic acid comprises a recognition site for the recombinase and a cargo nucleic acid flanked by at least one transposon end sequence.

The CRISPR-Cas system and the transposon system may be on the same or different vector(s). The recombinase, or catalytic domain thereof, is on the same or different vector(s) from the CRISPR-Cas system and/or the transposon system

The recombinase may comprise a tyrosine recombinase and/or a serine recombinase. In some embodiments, the recombinase comprises a Cre recombinase, a mutant, variant, or catalytic domain thereof and the recognition site comprises a lox site or variant thereof. In some embodiments, the recombinase comprises flippase (FLP) recombinase, a mutant, variant, or catalytic domain thereof and the recognition site comprises a flippase recognition target (FRT) site or variant thereof. In some embodiments, the recombinase comprises TniR resolvase, a mutant, variant, or catalytic domain thereof and the recognition site comprises a resolution (res) sequence or variant thereof. In some embodiments, the recombinase comprises a recombinase from a Tn3-like system (e.g., Tn3 resolvase), also known as TnpR, resolvase, a mutant, variant, or catalytic domain thereof and the recognition site comprises a resolution (res) sequence or variant thereof. Additional exemplary recombinases that find use in the methods, systems, and compositions of the invention are described herein. Homologs and enzymatically functional equivalents of any such recombinases may also be employed.

Also provided is a cell comprising the system described herein. In some embodiments, the cell is a eukaryotic cell.

Further provided is a method for resolving cointegration products resulting from RNA-guided nucleic acid integration, wherein the method comprises introducing into a cell: an engineered CRISPR-Cas system, and/or one or more vectors encoding the engineered CRISPR-Cas system, wherein the CRISPR-Cas system comprises: (a) at least one Cas protein, and (b) a guide RNA (gRNA); an engineered transposon system, and/or one or more vectors encoding the engineered transposon system, wherein the transposon system is configured for replicative (copy-and-paste) transposition; a recombinase, or catalytic domain thereof, and/or one or more vectors encoding a recombinase, or catalytic domain thereof; and at least one donor nucleic acid to be integrated, wherein the donor nucleic acid comprises a recognition site for the recombinase and a cargo nucleic acid flanked by at least one transposon end sequence, wherein the cell comprises a nucleic acid sequence with a target site and the donor nucleic acid is integrated downstream of the target site.

In some embodiments, the recombinase, or catalytic domain thereof, and/or one or more vectors encoding a recombinase, or catalytic domain thereof is introduced to the cell after the introduction of the engineered CRISPR-Cas system, the engineered transposon system, and the at least one donor nucleic acid.

In some embodiments, the CRISPR-Cas system and the transposon system are on the same or different vector(s). In some embodiments, the recombinase, or catalytic domain thereof, is on the same or different vector(s) from the CRISPR-Cas system and/or the transposon system.

The recombinase may comprise a tyrosine recombinase and/or a serine recombinase. In some embodiments, the recombinase comprises a Cre recombinase, a mutant, variant, or catalytic domain thereof and the recognition site comprises a lox site or variant thereof. IN some embodiments, the recombinase comprises flippase (FLP) recombinase, a mutant, variant, or catalytic domain thereof and the recognition site comprises a flippase recognition target (FRT) site or variant thereof. In some embodiments, the recombinase comprises TniR resolvase, a mutant, variant, or catalytic domain thereof and the recognition site comprises a resolution (res) sequence or variant thereof. In some embodiments, the recombinase comprises a recombinase from a Tn3-like system (e.g., Tn3 resolvase), also known as TnpR, a mutant, variant, or catalytic domain thereof and the recognition site comprises a resolution (res) sequence or variant thereof. Additional exemplary recombinases that find use in the methods, systems, and compositions of the invention are described herein. Homologs and enzymatically functional equivalents of any such recombinases may also be employed.

In some embodiments, the step of introducing into a cell comprises administering to a subject. In some embodiments, the administering comprises intravenous administration.

Kits comprising any or all of the components of the systems described herein are also provided. In some embodiments, the kit further comprises one or more reagent, shipping and/or packaging containers, one or more buffers, a delivery device, instructions, or a combination thereof. The delivery device may include at least one of an infusion device, an intravenous solution bag, a hypodermic needle, and a syringe.

Other aspects and embodiments of the disclosure will be apparent in light of the following detailed description.

### BRIEF DESCRIPTION OF THE DRAWINGS

FIGS. 1A-1C are schematics of the mechanisms and genetic architectures of Tn7 and Tn7-like transposons. FIG. 1A illustrates that the well-studied E. coli Tn7 transposon mobilizes via two mutually exclusive pathways. In the TnsABC+TnsD pathway (FIG. 1A left), TnsD binds to a conserved genomic attachment site known as attTn7, leading to sequence-specific integration of the transposon donor at a site adjacent to attTn7. In the TnsABC+TnsE pathway (FIG. 1A right), TnsE recognizes the lagging strand replication fork of replicating mobile plasmids, leading to sequence-non-specific integration. The TnsABCDE proteins are encoded by the genes tnsA-tnsB-tnsC-tnsD-tnsE found within the Tn7 transposon. RNA-guided DNA integration by Tn7-like transposons that harbor CRISPR-Cas systems mobilize similarly to the TnsABC+TnsD pathway, except TnsD is replaced by the TniQ homolog, and target recognition is facilitated by the RNA-guided CRISPR-associated complex. FIG. 1B shows the transposon-associated genes encoded by Tn7 (bottom) and the related transposons shown, which harbor conserved left (L) and right (R) transposon ends, as well as genes that are presumably responsible for transposition. The tnsA gene encodes an endonuclease-like protein that is responsible for introducing breaks at the 5′ ends of both strands during transposon excision and is absent in Tn5090/Tn5053. tnsB and the tniA homolog encode the integrase/transposase responsible for both 3′-end excision and integration reactions. tnsC and the tniB homolog encode AAA+ATPase regulator proteins, and the other genes shown encode proteins involved in target selection. The tniR gene in Tn5090/Tn5053 encodes a site-specific recombinase that acts at the element resolution site (res), and resolves the cointegrates formed during replicative transposition of the Tn5090/Tn5053, since the absence of the tnsA endonuclease gene means that simple cut-and-paste transposition is not possible. FIG. 1C illustrates exemplary bond breakage and joining reactions for Tn7 transposition. TnsA cleaves at the 5′ ends of the transposon, whereas TnsB cleaves at the 3′ ends of the transposon, during the excision step. TnsB is also the integrase that catalyzes concerted transesterification reactions, whereby the free 3′-OH ends of the excised transposon attack the phosphodiester bonds of both strands of the target DNA, leading to gapped ends that are filled in by endogenous DNA polymerase during subsequent steps. In the case of transposons that lack the tnsA gene, this mechanism of cut-and-paste transposition is not possible, the transposon must instead by mobilized through replicative transposition involving a Shapiro intermediate and a co-integrate product. These figure panels are adapted from FIG. 1 of Peters et al., Mol Microbiol 93, 1084-1092 (2014), incorporated herein by reference in its entirety. GCGGG is SEQ ID NO: 44 and CGCCC is SEQ ID NO: 45

FIGS. 2A and 2B are schematics of transposition via cut-and-paste versus copy-and-paste (replicative) mechanisms. The well-studied E. coli Tn7 transposon mobilizes via a cut-and-paste mechanism (FIG. 2A). TnsA and TnsB cleave both strands of the transposon DNA at both ends, leading to clean excision of a linear dsDNA, which contains short 3-nucleotide 5′-overhangs on both ends (not shown). The free 3′-OH ends are then used as a nucleophile by TnsB to attack phosphodiester bonds on both strands of the target DNA, resulting in concerted transesterification reactions. After gap fill-in, the transposition reaction is complete, and the integrated transposon is flanked by 5-bp target site duplications (TSD) on both ends as a result of the gap fill-in reaction. Some transposons instead mobilize via a copy-and-paste pathway (FIG. 2B), also known as replicative transposition. This results when the 5′ ends of the transposon donor DNA are not broken during the excision step, as is the case when the tnsA endonuclease gene is absent from the gene operon encoding the transposition proteins. In this case, the 3′-OH ends are still liberated and can participate in staggered transesterification reactions with the target DNA (FIG. 2B inset, middle right), catalyzed by TnsB, but the 5′ ends of the transposon remain covalently linked to the remainder of the DNA within the donor DNA molecule, which can be a genome or a plasmid vector. This copy-and-paste reaction results in what's known as a Shapiro intermediate (FIG. 2B middle), in which the entirety of the donor DNA—including the transposon sequence itself, as well as the flanking sequences—is joined together with the broken target DNA. This intermediate can only be resolved during subsequent DNA replication (FIG. 2B bottom left), which results in a so-called cointegrate product. This cointegrate harbors two copies of the transposon itself (orange rectangle), flanked by the TSD on one side. Importantly, the cointegrate also harbors the entirety of the donor DNA molecule, as well as the entirety of the target DNA molecule. Thus, in cases where the transposon is encoded on a plasmid vector, the entirety of the vector is joined to the target DNA during replicative transposition. At some frequency, the cointegrate product can be resolved into the products shown at the right, either through the action of a dedicated resolvase protein (e.g., the TniR protein in Tn5090/Tn5053, or the TnpR protein in Tn3), or through endogenous homologous recombination because of extensive homology between the two copies of the transposon itself in the cointegrate product. Cointegrate resolution results in a target DNA harboring a single transposon flanked by the TSD, as well as a regenerated version of the donor DNA molecule. These figure panels are adapted from FIGS. 1 and 2 of Hickman, A. B. & Dyda, F. DNA Transposition at Work. Chem Rev 116, 12758-12784 (2016), incorporated herein by reference in its entirety.

FIG. 3 is schematics of representative Type I-F and Type V-K CRISPR-Cas systems. A representative Tn7-like transposon that harbors a Type I-F variant CRISPR-Cas system is shown in the top schematic. Hallmark genes of a Type I-F variant CRISPR-Cas systems encode a Cascade complex; the Tn6677 transposon from Vibrio cholerae that mediates RNA-guided DNA insertion is a member of this family. Note the similarities in the transposition genes found in Tn6677 and related transposons and Tn7: the tnsA-tnsB-tnsC operon is maintained, whereas the tnsD homolog known as tniQ is encoded within the operon that encodes the Cas8-Cas7-Cas6 proteins that collectively form the RNA-guided TniQ-Cascade complex. The TnsA and TnsB protein products mediate transposon excision, whereas TnsB mediates integration of the transposon into the target DNA. A representative Tn7-like transposon that harbors a Type V-K CRISPR-Cas system systems is shown in the lower schematic. The hallmark gene of a Type V-K CRISPR-Cas system encodes Cas12k (also known as C2c5). Whereas tnsB, tnsC, and tniQ genes are present in these transposons, the tnsA gene is absent, indicating that these transposons do not encode the necessary machinery to mediate cut-and-paste transposition. Instead, they are likely to proceed via copy-and-paste replicative transposition, resulting in a cointegrate product rather than a clean integration product.

FIG. 4 is multiple sequence alignments of TnsB from different classes of Tn7-like transposons. The TnsB protein from three different transposons was compared: the E. coli Tn7 transposon, which does not harbor a CRISPR-Cas system (top); the V. cholerae Tn6677 transposon which harbors a variant Type I-F CRISPR-Cas system (middle); and the Scytonema hofinanni transposon which harbors a Type V-K CRISPR-Cas system (bottom). The three proteins were subjected to HHpred analysis which revealed strong conservation between all three proteins. Specifically, three core protein families (Pfam) are denoted in black, grey, and light grey lines that are common to all three proteins, and which cover similar regions of each protein's primary amino acid sequence. Additional Pfam IDs are detected by HHpred but are not displayed here. Based on this analysis and other analyses not shown, one can exclude the possibility that the TnsB from Tn7-like transposons that harbor Type V-K CRISPR-Cas systems possesses distinct chemical/enzymatic capabilities as compared to TnsB from Tn7 and Tn6677. Specifically, one can reasonably conclude that the TnsB protein in all three transposon classes cleaves the 3′ strand of the transposon ends, but not the 5′ strands.

FIGS. 5A-5C are schematics of the strategies for driving resolution of transposition cointegrate products using resolvases or recombinases. FIG. 5A illustrates replicative ‘copy-and-paste’ transposition by transposons that harbor Type V-K CRISPR-Cas systems. One embodiment of an expression strategy for the RNA and protein components is shown (left), in which a single-guide RNA (sgRNA) is driven by one promoter, and expression of the Cas12k, TniQ, TnsB, and TnsC proteins is controlled by a second promoter—both expression cassettes are encoded on a single plasmid termed pCasTns. The mini-transposon containing a genetic cargo of interest (cargo) is encoded on a second plasmid, termed pDonor, which the cargo is flanked by the conserved left (L) and right (R) transposon ends. The remainder of the donor DNA vehicle is denoted in red here. Introduction of both plasmid components in the present of a target DNA that contains a site complementary to the sgRNA—the target DNA may be genomic DNA, or can be encoded on other vector—leads to site-specific transposition in which the transposon is inserted at a distance downstream of the site targeted by the Cas12k-sgRNA complex. Because the transposon does not contain the TnsA endonuclease component, the transposition product is the result of replicative copy-and-paste transposition: the mini-transposon is present as a tandem duplicate, flanking the remainder of the entire donor vector sequence (shown in red). Resolution of this cointegrate product may occur by homologous recombination, but this process is generally low efficiency, and will be restricted to cells in which homologous recombination factors are expressed. Note that this scheme, and the schemes shown in FIGS. 5A-5C, may represent transposition taking place in bacterial cells, archaeal cells, or eukaryotic cells; the choice of promoter and vector will depend upon the application of interest. In other embodiments, the protein and RNA components may be expressed off of more or fewer different plasmids; from different types of promoters; the mini-transposon may be present on other vectors, or be constructed differently. FIG. 5B illustrates one embodiment of the system described herein, in which the RNA-guided DNA integration system containing a Type V-K CRISPR-Cas system is combined with the TniR resolvase and the resolution (res) sequence it recognizes, to allow for highly efficient TniR-mediated resolution of cointegrate products into the desired final transposition product. The initial replicative transposition pathway occurs as described above for panel FIG. 5A, but TniR can specifically act upon the cointegrate product because the duplicated transposon now presents two tandem copies of the res. TniR then mediates recombination between both res sequences, resulting in elimination of the donor DNA vehicle sequence (red) as well as the duplicate copy of the mini-transposon. FIG. 5C illustrates another embodiment of the system described herein utilizing a similar approach to high efficiency resolution of cointegrate products as described in FIG. 5B, but where Cre-Lox is used to effect recombination of cointegrate into the desired transposition product. The cointegrate leaves two tandem copies of the loxP sites that can be acted upon by the Cre recombinase for a targeted deletion of the duplicate transposon and the remainder of the donor DNA vector sequence. Note that for panels FIGS. 5B and 5C, the TniR/Cre proteins may be encoded on the same pCasTns vector or on a different vector; may be introduced in a separate transformation/transfection step; may be encoded within the same operon as the Cas-Tns proteins; or may be expressed in yet another embodiment other than those described above. In embodiments not shown, the Cre-Lox recombination system can be replaced with the FLP-FRT recombination system, or yet another recombination/resolution system that functions analogously.

FIGS. 6A-6D show the formation of cointegrates using Tn6677 TnsA D90A mutant. FIG. 6A is a schematic of the replicative ‘copy-and-paste’ transposition by transposons harboring Type-I CRISPR-Cas systems, where TnsA is mutated and lacks 5′ strand cleavage activity. One embodiment of an expression strategy for the RNA and protein components is shown (left), in which expression of the CRISPR and Cas8, Cas7, Cash, TniQ, TnsA, TnsB, and TnsC proteins is controlled by a single promoter within the plasmid termed pEffector, and where TnsA contains a D90A active site mutation. The mini-transposon containing a genetic cargo of interest (cargo) is encoded on a second plasmid, termed pDonor, which the cargo is flanked by the conserved left (L) and right (R) transposon ends. The remainder of the donor DNA vehicle is denoted in red. Introduction of both plasmids results in site-specific DNA integration as previously described. Because TnsA lacks 5′ strand cleavage capability, the transposition product is the result of replicative copy-and-paste transposition: the mini-transposon is present as a tandem duplicate, flanking the remainder of the entire donor vector sequence (shown in red). FIG. 6B is an alignment of exemplary sequencing reads from single-molecule real-time (SMRT) long-read, whole-genome sequencing of E. coli cell samples where site-specific DNA integration was performed using the pEffector construct encoding the TnsA D90A mutant. Reads were aligned to a reference genome containing a cointegrate and supports the existence of genome-transposon-vector junctions characteristic of cointegrates. FIG. 6C is an alignment of exemplary reads from a similar experiment as described in FIG. 6B, but instead DNA integration was performed using a vector construct encoding the wildtype TnsA. Disagreement between the reads and the reference genome supports the lack of cointegrate formation when wildtype TnsA was used. FIG. 6D are graphs of the quantification of reads from the two experiments described above, showing the number of SMRT reads from each experiment supporting either simple genomic integration of the mini-transposon, or formation of cointegrates. While the wildtype TnsA can only form simple insertions, TnsA D90A mutant results in a mix of cointegrates as well as a smaller number of simple insertions, likely from inefficient homologous recombination of cointegrate products.

FIGS. 7A-7C show the formation of cointegrates using transposons encoding wildtype Type V-K CRISPR-Cas systems. FIG. 7A is an alignment of exemplary sequencing reads from SMRT long-read, whole-genome sequencing of E. coli cell samples where site-specific DNA integration was performed using a vector construct encoding protein machinery for the Type V-K CRISPR-Cas transposon system found in S. hofinannii PCC 7110, and a separate vector containing the respective mini-transposon. Reads were aligned to a reference genome containing a cointegrate and supports the existence of genome-transposon-vector junctions characteristic of cointegrates. FIG. 7B is an alignment of exemplary reads from a similar experiment as described in FIG. 7A, but instead site-specific DNA integration was performed using a vector construct encoding protein machinery for the ShCAST (an exemplary Tn7-like transposon) Type V-K CRISPR-Cas transposon system, and a separate vector containing the respective mini-transposon. Reads were aligned to a reference genome containing a cointegrate and supports the existence of genome-transposon-vector junctions characteristic of cointegrates. FIG. 7C are graphs of the quantification of reads from the ShCAST experiments described in FIG. 7B, where site-specific DNA integration was performed with either sgRNA-252 or sgRNA-261, showing the number of SMRT reads from each experiment supporting either simple genomic integration of the mini-transposon, or formation of cointegrates.

### DETAILED DESCRIPTION

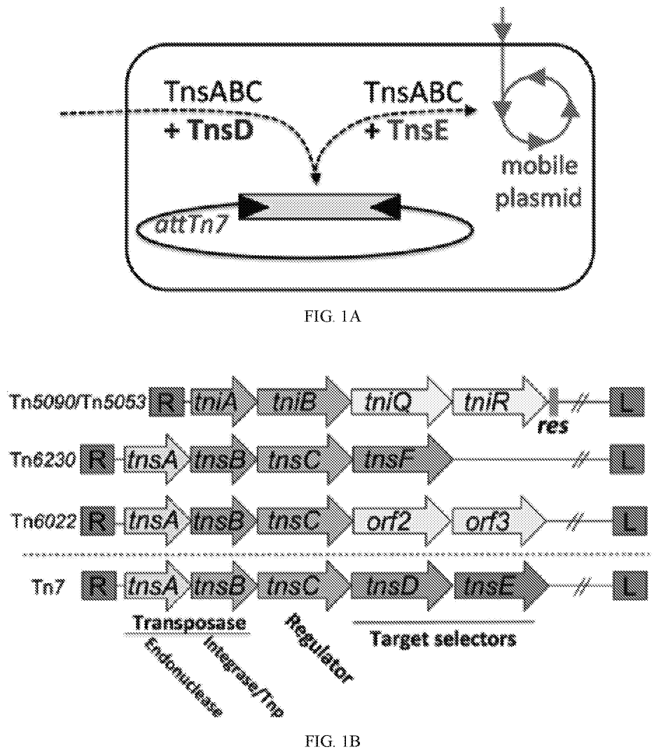

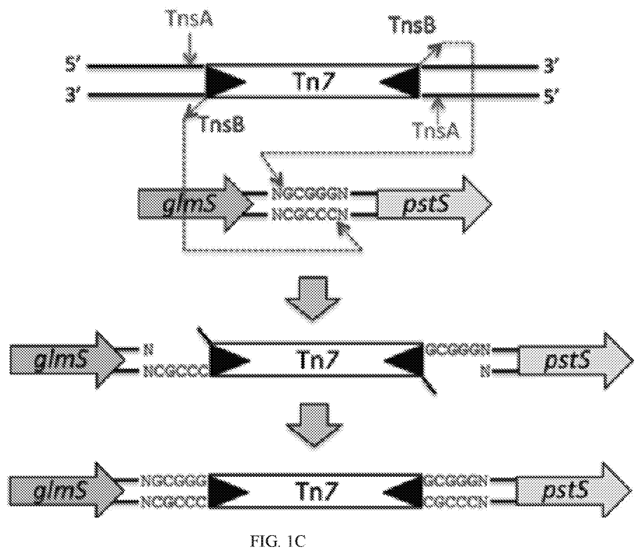

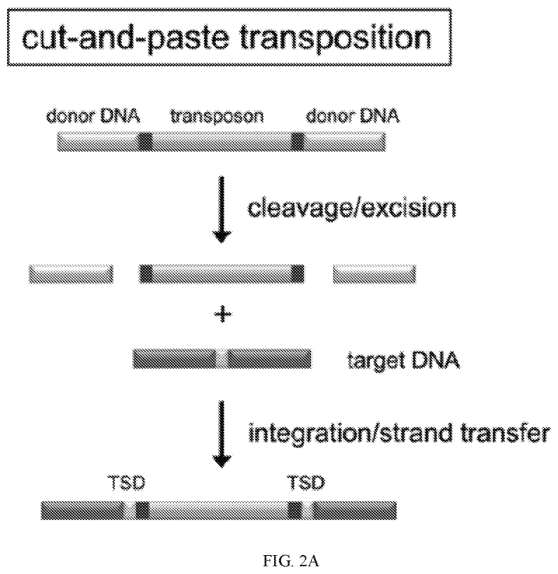

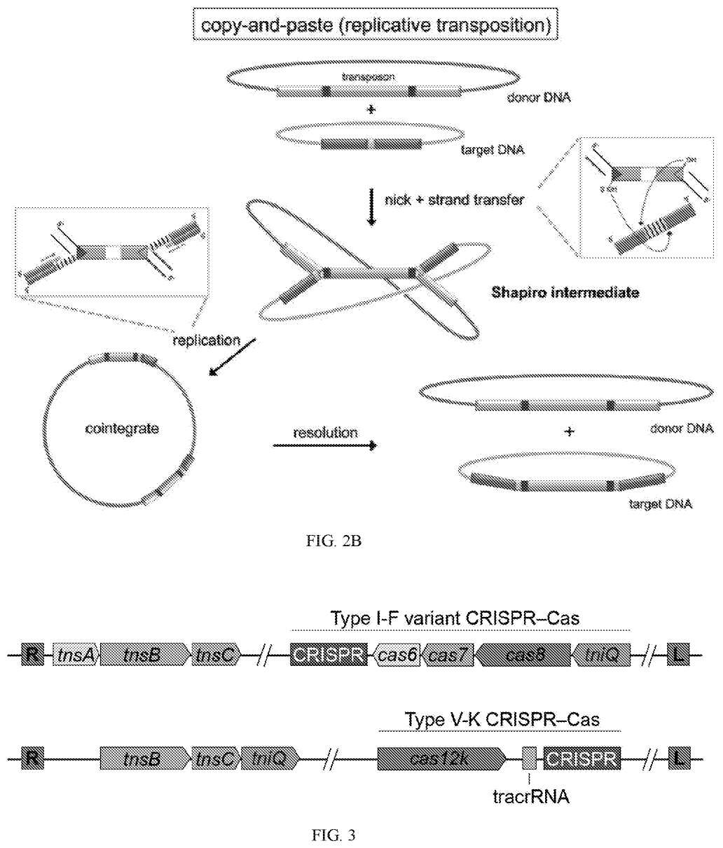

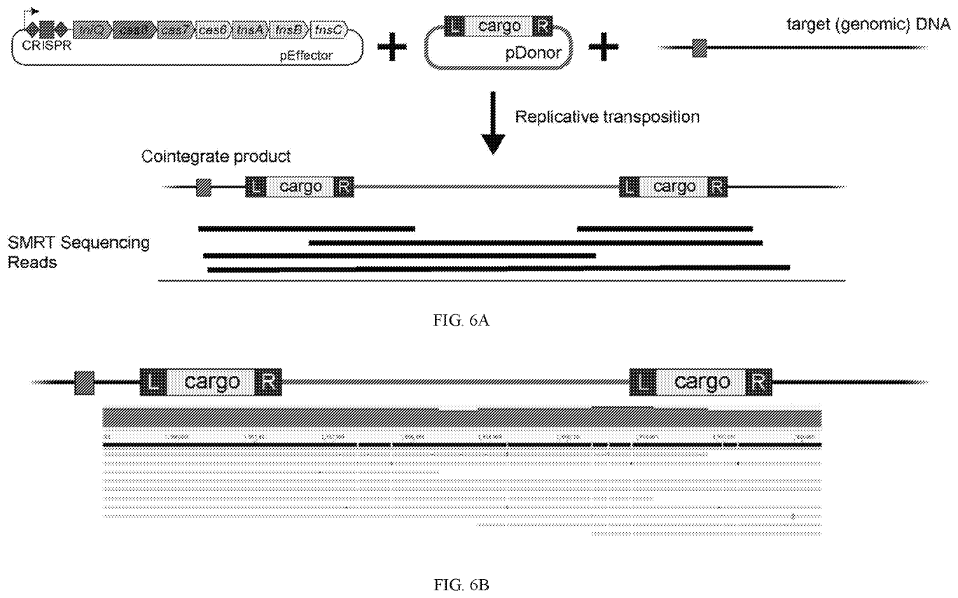

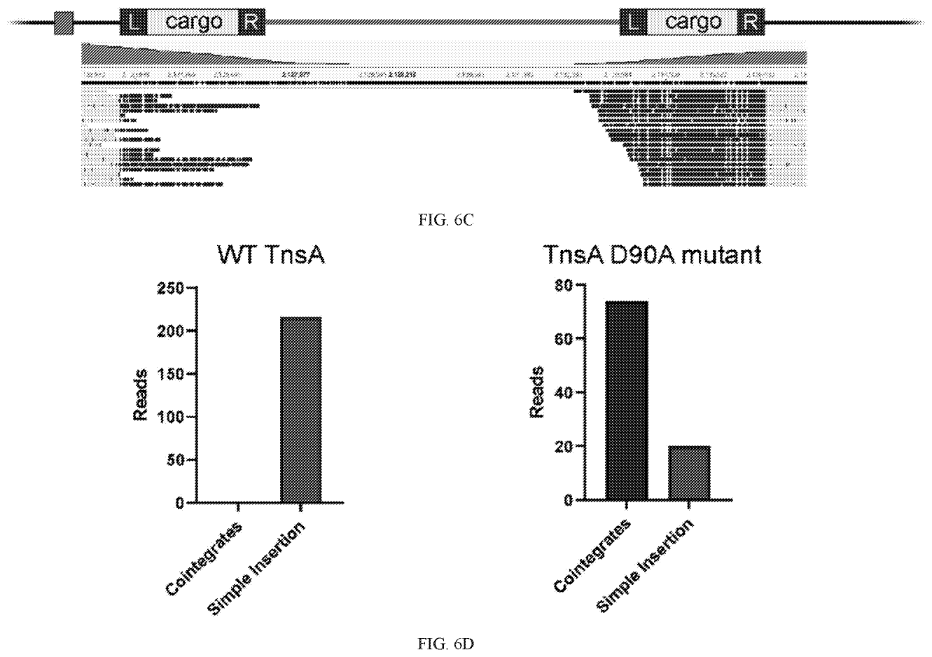

The disclosed systems, kits, and methods advance RNA-guided nucleic acid modification by resolving cointegrate products. 100251 Programmable integrases whose sequence specificity is governed exclusively by guide RNAs was recently described (Klompe, S. E., et al., Nature 571, 219-225 (2019), incorporated herein by reference in its entirety). A candidate CRISPR-transposon from Vibrio cholerae (Tn6677) was selected and RNA-guided transposition was reconstituted in an E. coli host. DNA integration occurred ˜47-51 base pairs (bp) downstream of the genomic site targeted by the CRISPR RNA (crRNA), and utilized the transposition proteins TnsA, TnsB, and TnsC, in conjunction with the RNA-guided DNA targeting complex TniQ-Cascade. Tn6677, like Tn7, naturally proceeded via a cut-and-paste mechanism of transposition, in which TnsA and TnsB catalyze 5′ and 3′ strand cleavage on both transposon ends, as has been demonstrated biochemically for Tn7 (FIGS. 1C and 2A). As shown herein, a D90A TnsA mutant, predicted to lack TnsA catalytic activity, catalyzed replicative, copy-and-paste transposition, in which a Shapiro intermediate led to eventual cointegrate formation because of the inability of TnsA to nucleolytically liberate the 5′ end of both transposon ends prior to the integration reaction being catalyzed by TnsB (FIGS. 6A-6D). In addition, using wild-type TnsA can produce a small amount of cointegration products.

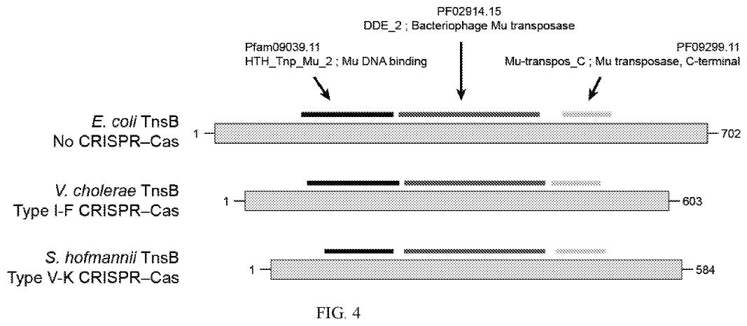

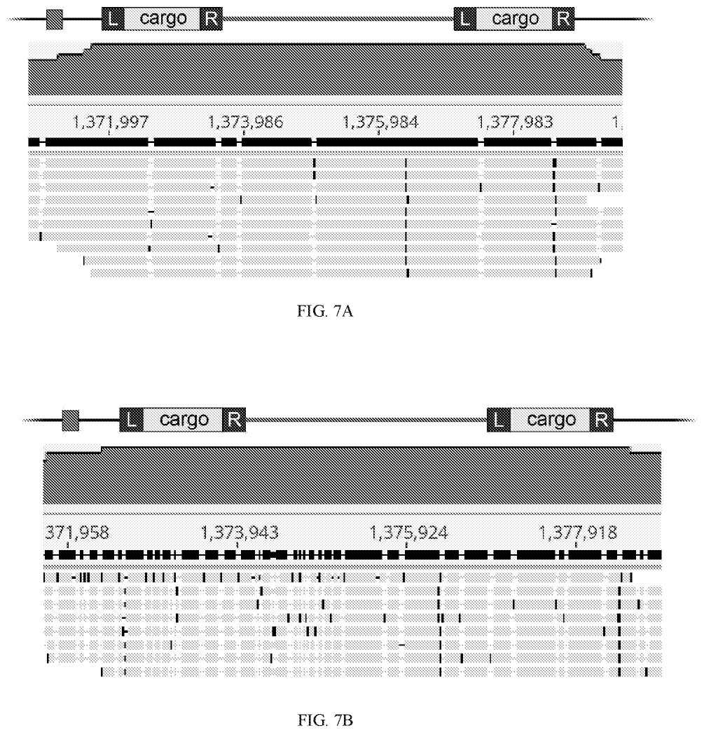

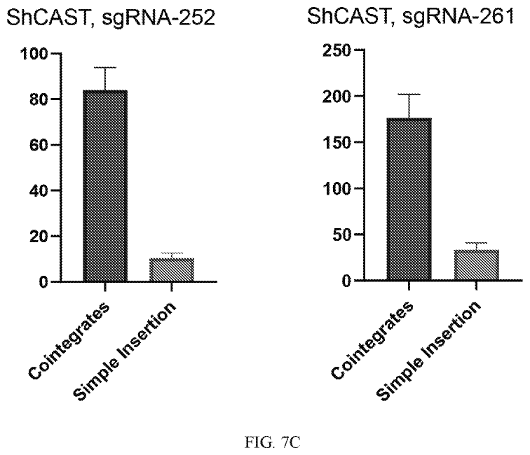

Remarkably, bacterial transposons have hijacked at least three distinct CRISPR-Cas subtypes. The Type V-K effector protein, Cas12k, also directed targeted DNA integration, albeit with lower fidelity. The CRISPR-Cas machinery implicated in a Type V-K process (FIG. 3) comprised the Cas12k protein and a dual-guide RNA (which could be fused into a single chimeric guide RNA, or sgRNA); the transposon-associated genes comprised tnsB-tnsC-tniQ (Strecker et al., Science 365, 48-53 (2019), incorporated herein by reference). The tnsA gene is entirely absent in the transposons that encode Type V-K CRISPR-Cas systems and the TnsB protein sequence encoded by the tnsB gene shows clear homology to both TnsB encoded by E. coli Tn7 and TnsB encoded by V. cholerae Tn6677 (FIG. 4), indicating that the catalytic domains between the three TnsB homologs are all shared, and excluding the likelihood of the TnsB from S. hofinanni possessing additional domains that could catalyze new reactions beyond the excision and integration reactions previously document for E. coli TnsB. PCR assays could not initially discriminate between a cut-and-paste versus a copy-and-paste transposition mechanism for the Type V-K CRISPR-Cas system, since the boundary junctions across the transposon ends and genomic integration site would be the same with both a normal cut-and-paste transposition product as well as a cointegrate product resulting from replicative transposition. As shown herein, a system comprising a Type V-K CRISPR-Cas system and a transposon system having TniQ catalyzed replicative, copy-and-paste transposition (FIGS. 7A-7C).

Cointegrate transposition products are generally undesirable for genome engineering applications involving RNA-guided integrases, for a number of reasons. Because the transposition pathway involves replication, there is extensive need for DNA synthesis, as opposed to the very limited gap repair that is used during cut-and-paste transposition. The cointegrate product resulting from replicative transposition contains duplicated copies of the transposon, which means that any genetic payload desired for insertion will be inserted in two copies rather than one, complicating applications of the tool. The cointegrate product also contains the entire donor vehicle inserted into the target site, which means that any sequences on this donor vehicle—whether they be bacterial origins of replication and/or antibiotic resistance genes, as would be commonly found on plasmids propagated in E. coli; or virally derived sequences if the donor vehicle were provided by a viral vector such as recombinant adeno-associated virus (rAAV)—will be faithfully inserted into the genomic target site, leading to unwanted insertion of payloads other than the desired payload found within the boundaries of the transposon. Last but not least, resolution of the cointegrate into the desired, clean transposition product containing just a single copy of the transposon at the target site, without any residual sequences from the remainder of the donor vehicle, does not occur with 100% efficiency, and will occur stochastically and with variable efficiencies depending on the cell type, cell cycle, and other factors that are not controllable by the researcher.

Because resolution of the cointegrate occurs through homologous recombination, it would have a need for DNA repair factors that mediate homologous recombination, thereby excluding some of the very advantages of RNA-guided integrases over existing methods that rely on nucleases and homology-directed repair, namely, an avoidance of reliance on host repair factors.

In certain embodiments, it would be desirable to leverage Tn7-like transposons that harbor Type V-K CRISPR-Cas systems for RNA-guided DNA integration, or Tn7-like transposons that harbor Type I-F variant CRISPR-Cas systems with a catalytic TnsA mutant, and thus direct replicative (copy-and-paste) transposition. Some applications benefit from copy-and-paste transposition, because unlike cut-and-paste transposition, where the genetic payload flanked by transposon ends is excised from the donor site, copy-and-paste (replicative) transposition does not excise the transposon from the donor site. Thus, for some applications, it is desirable for the transposon to remain in the original donor vector once transposition and cointegrate resolution occurs, and for the total copy number of the transposon to increase for each insertion reaction, rather than stay the same. Examples include, but are not limited to, applications involving the spread of a transposon-based gene drive construct through a bacterial population, where the gene drive continuously spreads by inserting into new targets in both horizontally transferred plasmids as well as genomic loci. In order to facilitate efficient spreading within the population, it would be desirable to have continuously increasing transposon copy numbers, which is made possible by having a transposon product at both the new target site as well as the original donor site (copy-and-paste, instead of cut-and-paste). However, it would be desired for cointegrate products to be resolved at the new target site, rather than vector sequences and duplicated transposons persisting at every new target site. Although a donor site with a fully excised transposon can still be repaired by homologous recombination with a sister chromosome or sister plasmid to reinstate the transposon during cut-and-paste transposition (as described in Hagemann and Craig, Genetics 1193 January; 133(1):9-16, incorporated herein by reference in its entirety), this process can be inefficient or non-existent in certain species or cell types. Therefore, there is a need for DNA integration methods that leverage these transposons, but in a way that results in uniform conversion of the cointegrate product into a resolved product that contains just a single inserted transposon flanked by the 5-bp target site duplication, but without the flanking donor DNA sequences. In addition, while replicative transposition may be beneficial, it would still be important to resolve the cointegrate structure, due to the reasons discussed above.

Herein, transposons harboring CRISPR-Cas systems were paired with resolvases or other recombinases to drive resolution of transposition cointegrate products (e.g. from replicative (copy-and-paste) transposition).

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclature used in connection with, and techniques of cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

A “vector” or “expression vector” is a replicon, such as plasmid, phage, virus, or cosmid, to which another DNA segment, e.g. an “insert,” may be attached or incorporated so as to bring about the replication of the attached segment in a cell.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the terms “providing”, “administering,” “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

### System for RNA-Guided DNA Integration

Disclosed herein are systems or kits for RNA-guided DNA integration comprising: an engineered CRISPR-Cas system, and/or one or more vectors encoding the engineered CRISPR-Cas system, wherein the CRISPR-Cas system comprises: (a) at least one Cas protein, and (b) a guide RNA (gRNA); an engineered transposon system, and/or one or more vectors encoding the engineered transposon system, wherein the engineered transposon system is configured for replicative transposition; a recombinase, or catalytic domain thereof, and/or one or more vectors encoding a recombinase, or catalytic domain thereof; and at least one donor nucleic acid to be integrated, wherein the donor nucleic acid comprises a recognition site for the recombinase and a cargo nucleic acid flanked by at least one transposon end sequence.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell, a cell of a non-human primate, or a human cell. In some embodiments, the cell is a plant cell.

a. Recombinase

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3 (also known as TnpR), β-six, CinH, ParA, γδ, Bxb1, ϕC31, TP901, TG1, ϕBT1, R4, ϕRV1, ϕFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange. The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each are hereby incorporated by reference in their entirety). Other examples of recombinases that are useful in the methods and compositions described herein are known to those of skill in the art, and any new recombinase that is discovered or generated is expected to be able to be used in the different embodiments of the invention. In some embodiments, the recombinase is a serine recombinase. In some embodiments, the recombinase is a tyrosine recombinase.

In some embodiments, the catalytic domains of a recombinase are fused to another protein or provided alone. Recombinases such as this are known, and include those described by Klippel et al., EMBO J. 1988; 7: 3983-3989: Burke et al., Mol Microbiol. 2004; 51: 937-948; Olorunniji et al., Nucleic Acids Res. 2008; 36: 7181-7191; Rowland et al., Mol Microbiol. 2009; 74: 282-298; Akopian et al., Proc Natl Acad Sci USA. 2003; 100: 8688-8691; Gordley et al., J Mol Biol. 2007; 367: 802-813; Gordley et al., Proc Natl Acad Sci USA. 2009; 106: 5053-5058; Arnold et al., EMBO J. 1999; 18: 1407-1414; Gaj et al., “Proc Natl Acad Sci USA. 2011; 108(2):498-503; and Proudfoot et al., PLoS One. 2011; 6(4):e19537; the entire contents of each are hereby incorporated by reference. For example, serine recombinases of the resolvase-invertase group, e.g., Tn3 and γδ resolvases and the Hin and Gin invertases, have modular structures with autonomous catalytic and DNA-binding domains (See, e.g., Grindley et al., Ann Rev Biochem. 2006; 75: 567-605, the entire contents of which are incorporated by reference). The catalytic domains of these recombinases are thus amenable to being in protein fusions. Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases. Similarly, the core catalytic domains of tyrosine recombinases (e.g., Cre, integrase) are known, and can be similarly co-opted to engineer programmable site-specific recombinases as described herein.

In some embodiments, the recombinase comprises Cre recombinase, a mutant, variant or catalytic domain thereof and the recognition site is a Lox site or variant thereof. In certain embodiments, the Cre recombinase comprises an amino acid sequence of at least 70% identity (e.g. 75%, 80%, 85%, 90%, 95% or 99% identity) to SEQ ID NO: 1. In select embodiments, the Cre recombinase comprises an amino acid sequence of at least 70% identity to SEQ ID NO: 9. In some embodiments, the vector encoding the Cre recombinase comprises a nucleic acid sequence having at least 70% identity to SEQ ID NO: 10 or 11.

SEQ ID NO: 1
PKKKRKVSNLLTVHQNLPALPVDATSDEVRKNLMD
MFRDRQAFSEHTWKMLLSVCRSWAAWCKLNNRKWF
PAEPEDVRDYLLYLQARGLAVKTIQQHLGQLNMLH
RRSGLPRPSDSNAVSLVMRRIRKENVDAGERAKQA
LAFERTDFDQVRSLMENSDRCQDIRNLAFLGIAYN
TLLRIAEIARIRVKDISRTDGGRMLIHIGRTKTLV
STAGVEKALSLGVTKLVERWISVSGVADDPNNYLF
CRVRKNGVAAPSATSQLSTRALEGIFEATHRLIYG
AKDDSGQRYLAWSGHSARVGAARDMARAGVSIPEI
MQAGGWTNVNIVMNYIRNLDSETGAMVRLLEDGD
SEQ ID NO: 9
MGSSHHHHHHSSGLVPRGSHGGGSAAAMGTRLPKK
KRKVSNLLTVHQNLPALPVDATSDEVRKNLMDMFR
DRQAFSEHTWKMLLSVCRSWAAWCKLNNRKWFPAE
PEDVRDYLLYLQARGLAVKTIQQHLGQLNMLHRRS
GLPRPSDSNAVSLVMRRIRKENVDAGERAKQALAF
ERTDFDQVRSLMENSDRCQDIRNLAFLGIAYNTLL
RIAEIARIRVKDISRTDGGRMLIHIGRTKTLVSTA
GVEKALSLGVTKLVERWISVSGVADDPNNYLFCRV
RKNGVAAPSATSQLSTRALEGIFEATHRLIYGAKD
DSGQRYLAWSGHSARVGAARDMARAGVSIPEIMQA
GGWTNVNIVMNYIRNLDSETGAMVRLLEDGD

The recognition site for Cre recombinase may include any known Lox sequence or sequence variant, see for example, Missirlis, PI, et al., BMC Genomics, 7:73 (2006), incorporated herein by reference. In certain embodiments, the Lox site comprises a nucleic acid sequence of at least 70% identity to SEQ ID NO: 2.

SEQ ID NO: 2
ATAACTTCGTATAGCATACATTATACGAAGTTAT

In some embodiments, the recombinase comprises flippase (FLP) recombinase, a mutant, variant, or catalytic domain thereof and the recognition site is a flippase recognition target (FRT) site or variant thereof. In certain embodiments, the FLP recombinase comprises an amino acid sequence of at least 70% identity (e.g. 75%, 80%, 85%, 90%, 95% or 99% identity) to SEQ ID NO: 3. In some embodiments, the nucleic acid encoding the FLP recombinase comprises a nucleic acid sequence having at least 70% identity to SEQ ID NO: 12.

SEQ ID NO: 3
MPQFDILCKTPPKVLVRQFVERFERPSGEKIALCA
AELTYLCWMITHNGTAIKRATFMSYNTIISNSLSF
DIVNKSLQFKYKTQKATILEASLKKLIPAWEFTII
PYYGQKHQSDITDIVSSLQLQFESSEEADKGNSHS
KKMLKALLSEGESIWEITEKILNSFEYTSRFTKTK
TLYQFLFLATFINCGRFSDIKNVDPKSFKLVQN
KYLGVIIQCLVTETKTSVSRHIYFFSARGRI
DPLVYLDEFLRNSEPVLKRVNRTGNSSSNKQEYQL
LKDNLVRSYNKALKKNAPYSIFAIKNGPKSHIGRH
LMTSFLSMKGLTELTNVVGNWSDKRASAVARTTYT
HQITAIPDHYFALVSRYYAYDPISKEMIALKDETN
PIEEWQHIEQLKGSAEGSIRYPAWNGIISQEVLDY
LSSYINRRI

Several variant FRT sites exist (see Schlake T, et al., Biochemistry 33 (43): 12746-51 (1994), Senecoff J F, et al., Journal of Molecular Biology. 201 (2): 405-21 (1988) and Turan S, et al., Journal of Molecular Biology 402 (1): 52-69 (2010)) and are compatible with the systems and methods described herein. In certain embodiments, the FRT site comprises a nucleic acid sequence of at least 70% identity to SEQ ID NO: 4.

SEQ ID NO: 4
GAAGTTCCTATTCTCTAGAAAGTATAGGAACTTC

In some embodiments, the recombinase comprises TniR resolvase, a mutant, variant, or catalytic domain thereof and the recognition site comprises a resolution (res) sequence or variant thereof. In certain embodiments, the TniR resolvase comprises an amino acid sequence of at least 70% identity (e.g. 75%, 80%, 85%, 90%, 95% or 99% identity) to SEQ ID NO: 5. In some embodiments, the nucleic acid encoding the TniR resolvase comprises a nucleic acid sequence having at least 70% identity to SEQ ID NO: 13.

SEQ ID NO: 5
MLIGYMRVSKADGSQATDLQRDALIAAGVDPVHLY
EDQASGMREDRPGLTSCLKALRTGDTLVVWKLDRL
GRDLRHLINTVHDLTGRGIGLKVLTGHGAAIDTTT
AAGKLVFGIFAALAEFERELIAERTIAGLASARAR
GRKGGRPFKMTAAKLRLAMAAMGQPETKVGDLCQE
LGVTRQTLYRHVSPKGELRPDGEKLLSRI

The sequence of any known TniR res site may be used with the system and methods described herein. In certain embodiments, the res sequence comprises a nucleic acid sequence of at least 70% identity to SEQ ID NO: 6.

SEQ ID NO: 6
CGGCGAGAACTTTCTGGCTCACACTGTCACATAAT
CGAACGTATATGTGACAGGTACGAC

In some embodiments, the recombinase comprises a recombinase from a Tn3-like system (e.g., Tn3 resolvase), also known as TnpR, a mutant, variant, or catalytic domain thereof and the recognition site comprises a resolution (res) sequence or variant thereof. In certain embodiments, the Tn3-like resolvase comprises an amino acid sequence of at least 70% identity (e.g. 75%, 80%, 85%, 90%, 95% or 99% identity) to SEQ ID NO: 7. In some embodiments, the nucleic acid encoding a Tn3 resolvase comprises a nucleic acid sequence having at least 70% identity to SEQ ID NO: 14.

SEQ ID NO: 7
MRLFGYARVSTSQQSLDLQVRALKDAGVKANRIFT
DKASGSSTDREGLDLLRMKVEEGDVILVKKLDRLG
RDTADMIQLIKEFDAQGVAVRFIDDGISTDGDMGQ
MVVTILSAVAQAERRRILERTNEGRQEAKLKGIKF
GRRRTVDRNVVLTLHQKGTGATEIAHQLSIARSTV
YKILEDERAS

The sequence of any known Tn3-like resolvase res site may be used with the system and methods described herein (See e.g., Grindley ND, et al., Cell 30:19-27 (1982), incorporated herein by reference in its entirety). In certain embodiments, the res sequence comprises a nucleic acid sequence of at least 70% identity to SEQ ID NO: 8.

SEQ ID NO: 8
CCGTACGAAATGTTATAAATTATCGGACATCGTAA
AACTGTTACATTAATATGTCTATTAAATCGTAAAT
TTGTAATAATAGACATGAGTTGTCCGATATTCGAT
TTAAGGTACATTTTT

b. Donor DNA

The donor DNA may be a part of a bacterial plasmid, bacteriophage, plant virus, retrovirus, DNA virus, autonomously replicating extra chromosomal DNA element, linear plasmid, mitochondrial or other organellar DNA, chromosomal DNA, and the like. In some embodiments, the donor nucleic acid comprises a human nucleic acid sequence.

The donor DNA comprises a recognition site for the recombinase, described elsewhere herein, and a cargo nucleic acid flanked by at least one transposon end sequence. In some embodiments, the cargo nucleic acid comprises the recognition site for the recombinase. Put another way, the recognition site for the recombinase is within the cargo nucleic acid. The term “transposon end sequence” refers to any nucleic acid comprising a sequence capable of forming a complex with the transposase enzymes thus designating the DNA between these two ends for rearrangement. Usually these sequences are inverted repeats about 9 to 40 base pairs long, however the exact sequence requirements differ for the specific transposase enzymes. Transposon end sequences are well known in the art. Transposon ends sequences may or may not include additional sequences that promotes or augment transposition.

The donor DNA, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or less than 10 kb, in length or greater. The donor DNA, and the cargo nucleic acid, may be at least or about 10 kb, at least or about 50 kb, at least or about 100 kb, between 20 kb and 60 kb, between 20 kb and 100 kb.

c. CRISPR-Cas System

CRISPR-Cas systems have been successfully utilized to edit the genomes of various organisms, including, but not limited to bacteria, humans, fruit flies, zebra fish and plants.

In some embodiments, the present system may be derived from a Class 1 (e.g., Type I, Type III, Type VI) or a Class 2 (e.g. Type II, Type V, or Type VI) CRISPR-Cas system. In some embodiments, the present system may be derived from a Type I CRISPR-Cas system or a Type V CRISPR-Cas system.

For example, Type I Cascade complexes may be used in the present methods and systems. Type I CRISPR-Cas systems encode a multi-subunit protein-RNA complex called Cascade, which utilizes a crRNA (or guide RNA) to target double-stranded DNA during an immune response. Cascade itself has no nuclease activity, and degradation of targeted DNA is instead mediated by a trans-acting nuclease known as Cas3. Intriguingly, the Type I-F CRISPR-Cas systems and Type I-B CRISPR-Cas systems found within Tn7 transposons consistently lack the Cas3 gene, suggesting that these systems no longer retain any DNA degradation capabilities and have been reduced to RNA-guided DNA-binding complexes. Additionally, one of the core proteins used by Tn7 transposons for selection of DNA target sites for purposes of transposon mobility, TnsD (also known as TniQ), is conspicuously encoded by a gene sitting directly within the Cas gene operon in these systems, suggesting direct coupling or functional relationship between the Cascade complex encoded by Cas genes, and the transpososome enzymatic machinery encoded by Tn seven (Tns) transposase genes.

The system derived from Vibrio cholerae that harbors a Type I-F CRISPR-Cas system may be used in the present method. Other systems (for which the CRISPR-Cas systems are either categorized as Type I-F or I-B) may also be used in the present method. These include, without limitation, systems from Vibrio cholerae, Photobacterium ihopiscarium, Pseudoalteromonas sp. P1-25, Pseudoalteromonas ruthenica, Photobacterium ganghwense, Shewanella sp. UCD-KL21, Vibrio diazotrophicus, Vibrio sp. 16, Vibrio sp. F12, Vibrio splendidus, Aliivibrio wodanis, and Parashewanella spongiae.

The Type V systems that encode putative effector gene known as Cas 12k, formerly known as c2c5, may be used in the present methods and systems. The Type V systems encode a putative effector that may be a single protein functioning with a single gRNA. These may have different packaging size, assembly, nuclear localization, etc. Type V CRISPR-Cas systems fall within Class 2 systems, which rely on single-protein effectors together with guide RNA, and so it remains possible that the engineering strategies may be streamlined by using single-protein effectors like Cas12k, rather than the multi-subunit protein-RNA complexes encoded by type I systems, namely Cascade. These operons may be cloned into the same backbones.

The present system may comprise Cas12k. The present system may comprise Cas5, Cas6, Cas7 Cas8, or a combination thereof. In some embodiments, the Cas5 and Cas8 are linked as a functional fusion protein.

d. gRNA

The gRNA may be a crRNA, crRNA/tracrRNA (or single guide RNA, sgRNA).

The terms “gRNA,” “guide RNA” and “CRISPR guide sequence” may be used interchangeably throughout and refer to a nucleic acid comprising a sequence that determines the binding specificity of the CRISPR-Cas system. A gRNA hybridizes to (complementary to, partially or completely) a target nucleic acid sequence (e.g., the genome) in a host cell. The system may further comprise a target nucleic acid.

The gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31 , 32, 33, 34, 35, 36, 37, 38, 39, 40, 41, 42, 43, 44, 45, 46, 47, 48, 49, 50, 51 , 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81 , 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The target nucleic acid may be flanked by a protospacer adjacent motif (PAM). A PAM site is a nucleotide sequence in proximity to a target sequence. For example, PAM may be a DNA sequence immediately following the DNA sequence targeted by the CRISPR/Cas system.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems). In some embodiments, e.g., Type I systems, the PAM is on the alternate side of the protospacer (the 5′ end). Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as TTT, TTG, TTC, TTTT (SEQ ID NO: 39), etc.), NGG, NGA, NAG, NGGNG and NNAGAAW (W=A or T, SEQ ID NO: 40), NNNNGATT (SEQ ID NO: 41), NAAR (R=A or G), NNGRR (R=A or G), NNAGAA (SEQ ID NO: 42) and NAAAAC (SEQ ID NO: 43), where “N” is any nucleotide.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

e. Transposon System

An engineered transposon system of the present invention may comprise one or more transposases or other components of a transposon. The engineered transposon system facilitates cleavage of the target nucleic acid and subsequent insertion of the donor nucleic acid into the target nucleic acid. The engineered transposon system of the present invention may be derived from any of the known transposon systems and/or transposon components. The transposon systems and components may have different efficiency, different specificity, different transposon end sequences, and the like, but retain the capability to facilitate cleavage of the target nucleic acid and subsequent insertion of the donor nucleic acid into the target nucleic acid.

In some embodiments, the transposon is a Tn7 or Tn7-like transposon. Tn7 and Tn7-like transposons may be categorized based on the presence of the hallmark DDE-like transposase gene, tnsB (also referred to as tniA), the presence of a gene encoding a protein within the AAA+ATPase family, tnsC (also referred to as tniB), one or more targeting factors that define integration sites (which may include a protein within the tniQ family, also referred to as tnsD, but sometimes includes other distinct targeting factors), and inverted repeat transposon ends that typically comprise multiple binding sites thought to be specifically recognized by the TnsB transposase protein. In Tn7, the targeting factors, or “target selectors,” comprise the genes tnsD and tnsE. Based on biochemical and genetics studies, it is known that TnsD binds a conserved attachment site in the 3′ end of the glmS gene, directing downstream integration, whereas TnsE binds the lagging strand replication fork and directs sequence-non-specific integration primarily into replicating/mobile plasmids. Thus, Tn7 exhibits mobilization patterns that allow for both horizontal and vertical spread (FIG. 1A). In some embodiments, the transposon system comprises TnsB and TnsC.

The most well-studied member of this family of transposons is Tn7, hence why the broader family of transposons may be referred to as Tn7-like. “Tn7-like” term does not imply any particular evolutionary relationship between Tn7 and related transposons; in some cases, a Tn7-like transposon will be even more basal in the phylogenetic tree and thus Tn7 can be considered as having evolved from, or derived from, this related Tn7-like transposon.

Whereas Tn7 comprises tnsD and tnsE target selectors, related transposons comprise other genes for targeting (FIG. 1B): for example, Tn5090/Tn5053 encode a member of the tniQ family (a homolog of E. coli tnsD) as well as a resolvase gene tniR (see below); Tn6230 encodes the protein TnsF; and Tn6022 encodes two uncharacterized open reading frames orf2 and orf3; Tn6677 and related transposons encode variant Type I-F and Type I-B CRISPR-Cas systems that work together with TniQ for RNA-guided mobilization; and other transposons encode Type V-U5 CRISPR-Cas systems that work together with TniQ for random and RNA-guided mobilization. Any of the above transposon systems are compatible with the systems and methods described herein. In some embodiments, the transposons system comprises TniQ.

Wild-type Tn7 undergoes cut-and-paste transposition (FIGS. 1C and 2A; reviewed in Peters, J. E. Tn7. Microbiol Spectr 2, (2014), incorporated herein by reference), ultimately resulting in the entire transposon being integrated at the target site, flanked by a 5-base pair (bp) target site duplication. The transposition mechanism is known as ‘cut-and-paste,’ because the transposon copy is excised from its donor site (or cut out), before being pasted into the target site. Note that the donor DNA, post transposon excision, may in some cases be degraded (because of inability to repair the lesion), repaired by homologous recombination if other homologous DNA copies are present, or be repaired through other means, e.g. microhomology.

In some transposon systems, the donor DNA is mobilized via copy-and-paste transposition, also referred to as replicative transposition (FIG. 2B; May & Craig. Science 272, 401-404 (1996), incorporated herein by reference). In this pathway, transfer of the 3′ ends of the transposon donor DNA to the target DNA results in a fusion product that is termed a Shapiro intermediate, or strand transfer intermediate. Repair of this intermediate involves extensive DNA replication that can be tens of kilobases in scale, compared to the smaller repair of simple gaps that typically occurs during cut-and-paste transposition. Furthermore, eventual repair via this mechanism results in a circular cointegrate molecule, in which the entire donor sequence (defined as the entire DNA that is the source of the transposon, not just the transposon itself; this is often a plasmid) and target sequence (defined as the entire DNA that contains the target sequence; this is often a bacterial chromosome) are joined by two duplicate copies of the transposon. Other transposons, including bacteriophage Mu and Tn3, also mobilize via a similar replicative transposition pathway involving extensive DNA replication. As shown herein, the D90A TnsA mutant catalyzed copy-and-paste transposition, in which a Shapiro intermediate leads to eventual cointegrate formation because of the inability of TnsA to nucleolytically liberate the 5′ end of both transposon ends prior to the integration reaction being catalyzed by TnsB (FIGS. 1C, 2B and 6). In some embodiments, the engineered transposon system utilizes copy-and-paste or replicative transposition.

In some embodiments, the transposon system comprises an inactive TnsA or lacks TnsA. TnsA may comprise any mutation capable of promoting transposition but without 5′ strand cleavage. For example, a mutation at position D90 (e.g., D90A point mutant)in V. cholera TnsA, or equivalent active site residue in other species. One of skill in the art would be able to determine evidence of copy-and-paste transposition and cointegrate formation in a TnsA mutant using the mutant in transposition experiments followed by sequencing the resulting products, as shown in FIG. 6 for the D90A point mutant. Thus, transposon systems may be converted from a primarily cut-and-paste mechanism to replicative transposition by converting native TnsA to a mutant version.

Cointegrate transposition products may be resolved, at some frequency, depending on cell type/strain, into simpler products in which the target DNA contains just a single copy of the transposon, and the remaining donor sequence (e.g., sequences outside the transposon on the donor DNA that encoded the transposon) are removed. This process can occur via homologous recombination between the two duplicate copies of the transposon within the co-integrate, and thus, this resolution process involves DNA repair factors that mediate homologous recombination (FIG. 2B).

Alternatively, some transposons—both Tn7-like and other classes of bacterial transposons that are not related or much less related to Tn7—have evolved alternative strategies to resolve cointegrate products. For example, Tn5053 and Tn5053-like transposons encode an additional resolvase gene, tniR, which encodes a protein product that facilitates resolution of the cointegrate into a transposition product containing just a single transposon rather than the duplicated transposon plus flanking donor DNA sequences (Kholodii et al., Mol Microbiol 17, 1189-1200 (1995), incorporated herein by reference). The TniR protein is related to recombinases of the resolvase/invertase family, and is thought to recognize a region of DNA known as a res region, because of its role in promoting resolution, often catalyzed by an upstream resolvase gene. Classical res regions occupy ˜170 bp of DNA and contain three resolvase-binding sites that are made up of pairs of inverted repeats. Tn5053 contains these three sites, though its particular res region is unusual. Tn3 is another transposon family that similarly undergoes replicative transposition involving initial cointegrate formation followed by resolution at the res region catalyzed by a Tn3 resolvase (Stark, et al., Trends Genet. 5, 304-309 (1989), incorporated herein by reference). In some embodiments, the transposon system lacks a resolvase.

The present system might comprise the transposon Tn6677 in combination with a variant Type I-F CRISPR-Cas system (See, Klompe et al., Nature 571, 219-225 (2019) and International Patent Application No. PCT/US20/21568, each incorporated herein by reference in their entirety). The transposon-associated genes comprise tnsA-tnsB-tnsC as well as the tniQ gene that is in the same operon as cas8-cas7-cas6. The transposon Tn6677 may be derived from a Vibrio cholerae or other applicable species, for example those disclosed in International Patent Application No. PCT/US20/21568.

A type V-K CRISPR-Cas system was shown to direct RNA-guided transposition, though a considerable degree of random integration still occurred in this system. The CRISPR-Cas machinery comprises the Cas12k protein and a dual-guide RNA (which could be fused into a single chimeric guide RNA, or sgRNA); the transposon-associated genes comprise tnsB-tnsC-tniQ. The transposon may be derived from a Scytonema hofinanni isolate (FIG. 3). The present system might comprise the transposon comprising tnsB-tnsC-tniQ, e.g., as derived from Scytonema hofinanni, or other homologous transposons, in combination with a variant Type V-K CRISPR-Cas system.

f. Vectors

The engineered CRISPR-Cas system and the engineered transposon system may be on the same or different vector(s). The recombinase, or catalytic domain thereof, may be on the same or different vector(s) from either the CRISPR-Cas system and/or the transposon system. For example, the system described herein can be employed through expression of the recombinase in trans. The present system can be delivered to a subject or cell using one or more vectors (e.g., 1, 2, 3, 4, 5, 6, 7, 8, 9, or more vectors). One or more gRNAs (e.g., sgRNAs) can be in a single (one) vector or two or more vectors. The vector may also include a donor DNA. One or more Cas proteins and/or transposon proteins and/or recombinase can be in the same, or separate vectors. The present disclosure further provides engineered, non-naturally occurring vectors and vector systems, which can encode one or more components of the present system.

Vectors can be administered directly to patients (in vivo) or they can be used to manipulate cells in vitro or ex vivo, where the modified cells may be administered to patients. The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Conventional viral and non-viral based gene transfer methods can be used to introduce nucleic acids encoding components of the present system into cells, tissues or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, the requisite protein and RNA machinery may be expressed on the same plasmid as the transposon donor, so that the entire system is fully autonomous. The machinery guiding the DNA targeting and DNA integration may be encoded within the transposon itself, such that it can guide further mobilization autonomously, whether in the originally transformed bug, or in other bugs (e.g., in a conjugative plasmid context, in a microbiome context, etc.).

In certain embodiments, the requisite protein and RNA machinery may be expressed on two or more plasmids.

Promoters that may be used include T7 RNA polymerase promoters, constitutive E. coli promoters, and promoters that could be broadly recognized by transcriptional machinery in a wide range of bacterial organisms. The system may be used with various bacterial hosts. 100901 In certain embodiments, plasmids that are non-replicative, or plasmids that can be cured by high temperature may be used. The transposon, and transposon/CRISPR-associated machinery, may be removed from the engineered cells under certain conditions. This may allow for RNA-guided integration by transforming bacteria of interest, but then being left with engineered strains that have no memory of the plasmids used to facilitate RNA-guided DNA integration.

Drug selection strategies may be adopted for positively selecting for cells that underwent RNA-guided DNA integration. A transposon may contain one or more drug-selectable markers within the cargo. Then presuming that the original transposon donor plasmid is removed, drug selection may be used to enrich for integrated clones. Colony screenings may be used to isolate clonal events.

A variety of viral constructs may be used to deliver the present system (such as one or more Cas proteins and/or Tns proteins, gRNA(s), donor DNA, etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

The present disclosure also provides for DNA segments encoding the proteins disclosed herein, vectors containing these segments and host cells containing the vectors. The vectors may be used to propagate the segment in an appropriate host cell and/or to allow expression from the segment (i.e., an expression vector). The person of ordinary skill in the art would be aware of the various vectors available for propagation and expression of a cloned DNA sequence. In one embodiment, a DNA segment encoding the present protein(s) is contained in a plasmid vector that allows expression of the protein(s) and subsequent isolation and purification of the protein produced by the recombinant vector. Accordingly, the proteins disclosed herein can be purified following expression from the native transposon, obtained by chemical synthesis, or obtained by recombinant methods.

To construct cells that express the present system, expression vectors for stable or transient expression of the present system may be constructed via conventional methods as described herein and introduced into host cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′-and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the host cell, the vectors may be maintained as an autonomously replicating sequence or extrachromosomal element or may be integrated into host DNA.

In one embodiment, the donor DNA may be delivered using the same gene transfer system as used to deliver the Cas protein, the recombinase, and/or transposon system proteins (included on the same vector) or may be delivered using a different delivery system. In another embodiment, the donor DNA may be delivered using the same transfer system as used to deliver gRNA(s).

In one embodiment, the present disclosure comprises integration of exogenous DNA into the endogenous gene. Alternatively, an exogenous DNA is not integrated into the endogenous gene. The DNA may be packaged into an extrachromosomal, or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system (e.g., proteins, polynucleotides encoding these proteins, donor polynucleotides and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan 1;459(1-2):70-83), incorporated herein by reference.

Exemplary vectors encoding the systems described herein are provided in SEQ ID NO: 15-38 and additional vectors appropriate for the methods and uses described herein may be found in International Application No. PCT/US20/21568.

### Methods

Also disclosed herein are methods for RNA-guided nucleic acid integration and resolving cointegration products from RNA-guided nucleic acid integration using the disclosed systems or kits.

The methods may comprise introducing into a cell: an engineered CRISPR-Cas system, and/or one or more vectors encoding the engineered CRISPR-Cas system, wherein the CRISPR-Cas system comprises: at least one Cas protein, and a guide RNA (gRNA); an engineered transposon system, and/or one or more vectors encoding the engineered transposon system, wherein the transposon system is configured for replicative transposition; a recombinase, or catalytic domain thereof, and/or one or more vectors encoding a recombinase, or catalytic domain thereof; and at least one donor nucleic acid to be integrated, wherein the donor nucleic acid comprises a recognition site for the recombinase and a cargo nucleic acid flanked by at least one transposon end sequence, wherein the cell comprises a nucleic acid sequence with a target site and the donor nucleic acid is integrated downstream of the target site. In some embodiments, the cell is ex vivo. In some embodiments, the introducing into a cell comprises administering to a subject.

The descriptions and embodiments provided above for the engineered CRISPR-Cas system, the engineered transposon system, the recombinase, or catalytic domain thereof, and the donor nucleic acid are applicable to the methods described herein.

In some embodiments, the introduction of the recombinase, or catalytic domain thereof, and/or one or more vectors encoding a recombinase, or catalytic domain thereof is simultaneous to the introduction of the CRISPR-Cas system, the transposon system, and the donor nucleic acid. For example, all four components may be introduced simultaneous or nearly simultaneously. In some embodiments, all four components may be introduced, in any order, with a time period separating each introduction. In alternative embodiments, the introduction of the recombinase to the cell after the introduction the CRISPR-Cas system, the transposon system, and the donor nucleic acid, such that RNA-guided nucleic acid integration has already occurred.

The method may comprise administering to the subject, in vivo, or by transplantation of ex vivo treated cells, a therapeutically effective amount of the described system. In some embodiments, the vector(s) is delivered to the tissue of interest by, for example, an intramuscular, intravenous, transdermal, intranasal, oral, mucosal, or other delivery methods. In some embodiments, administering comprises intravenous administration. Such delivery may be either via a single dose, or multiple doses.

The components of the present system or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

Administration may be through any suitable mode of administration, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, topical, and inhalation.

In some embodiments, an effective amount of the components of the present system or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful RNA-guided DNA integration is achieved and resolution of the co-integrate products is complete.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

The methods may be used for a variety of purposes as disclosed in International Application No. PCT/US20/21568, incorporated herein by reference in its entirety. For example, the methods may include, but are not limited to, inactivation of a microbial gene, RNA-guided DNA integration in a plant or animal cell, a method of treating a subject suffering from a disease or disorder (e.g., cancer, Duchenne muscular dystrophy (DMD), sickle cell disease (SCD), β-thalassemia, and hereditary tyrosinemia type I (HT1)), and methods of treating a diseased cell (e.g., a cell deficient in a gene which causes cancer).

### Kits

Also within the scope of the present disclosure are kits that include the components of the present system.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

The present disclosure also provides for kits for performing RNA-guided DNA integration in vitro. The kit may include the components of the present system. Optional components of the kit include one or more of the following: (1) buffer constituents, (2) control plasmid, (3) sequencing primers.

Polynucleotides/DNA containing the target site may include, but is not limited to, purified chromosomal DNA, total cDNA, cDNA fractionated according to tissue or expression state (e.g. after heat shock or after cytokine treatment other treatment) or expression time (after any such treatment) or developmental stage, plasmid, cosmid, BAC, YAC, phage library, etc. Polynucleotides/DNA containing the target site may include DNA from organisms such as Homo sapiens, Mus domesticus, Mus spretus, Canis domesticus, Bos, Caenorhabditis elegans, Plasmodium falciparum, Plasmodium vivax, Onchocerca volvulus, Brugia malayi, Dirofilaria immitis, Leishmania, Zea maize, Arabidopsis thaliana, Glycine max, Drosophila melanogaster, Saccharomyces cerevisiae, Schizosaccharomyces pombe, Neurospora, Escherichia coli, Salmonella typhimurium, Bacillus subtilis, Neisseria gonorrhoeae, Staphylococcus aureus, Streptococcus pneumonia, Mycobacterium tuberculosis, Aquifex, Thermus aquaticus, Pyrococcus furiosus, Thermus littoralis, Methanobacterium thermoautotrophicum, Sulfolobus caldoaceticus, and others.

### EXAMPLES

The following are examples of the present invention and are not to be construed as limiting.

### Example 1

### Resolving Cointegrate Products

Cointegrate products may result from the engineered transposon machinery alongside the Type V-K CRISPR-Cas systems, or any other system configured for replicative transposition, e.g. engineered transposon machinery comprises an inactive TnsA. As shown herein, a D90A TnsA mutant, predicted to lack TnsA catalytic activity, in combination with a Type I CRISPR-Cas system catalyzed replicative, copy-and-paste transposition, in which a Shapiro intermediate led to eventual cointegrate formation because of the inability of TnsA to nucleolytically liberate the 5′ end of both transposon ends prior to the integration reaction being catalyzed by TnsB (FIGS. 6A-6D). A system identical to this but having fully active wild-type TnsA also resulted in a small amount of cointegration product. Also shown herein, transpositions using Type V-K CRISPR-Cas systems with transposons encoding tnsD, tnsC, and tniQ, also overwhelming result in the formation of cointegrate products.

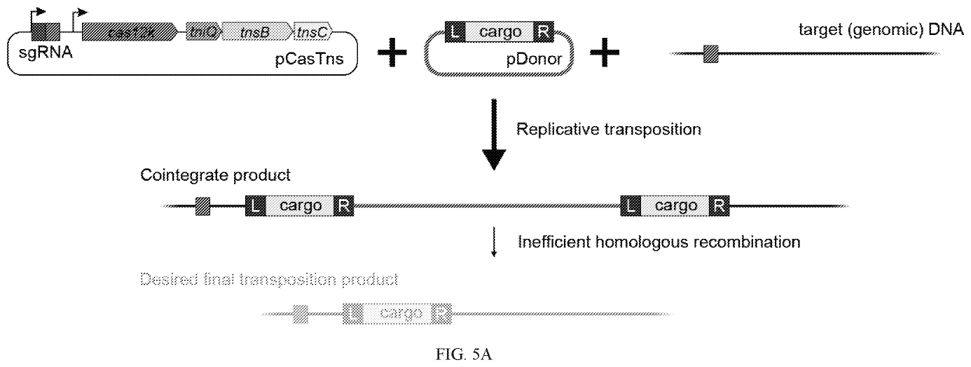

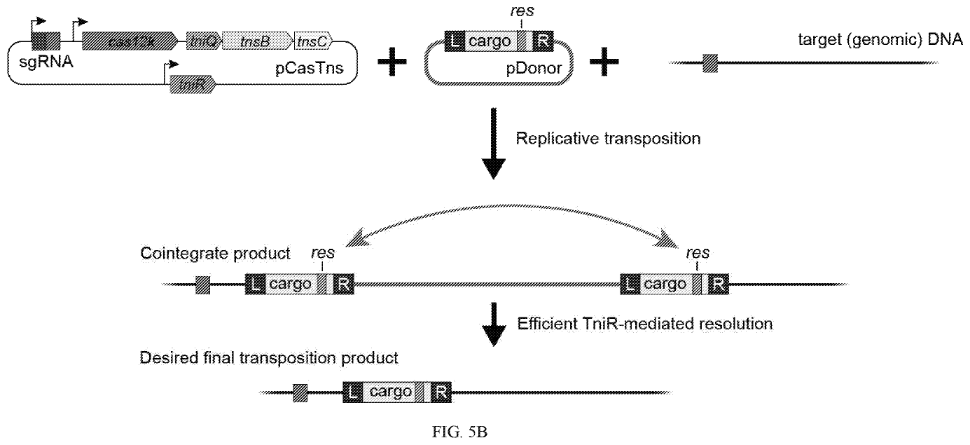

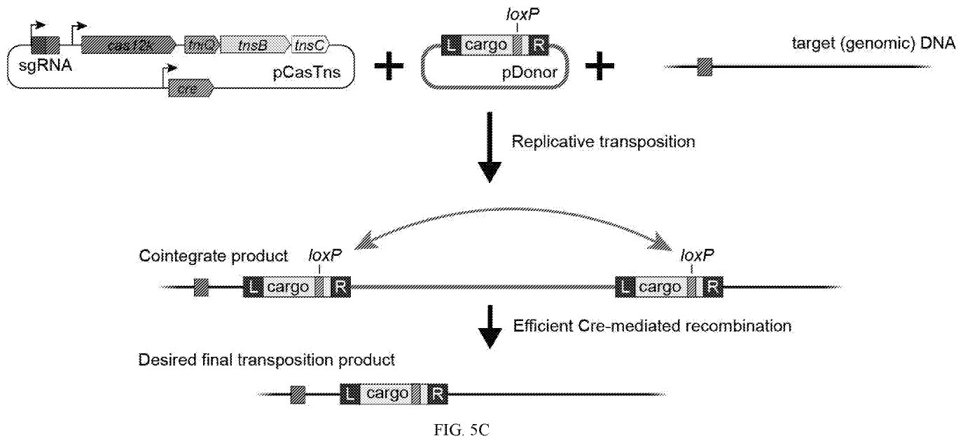

Herein cointegrate products are able to be resolved into single-transposon insertion products without the need for homologous recombination by leveraging natural resolvases or recombinases, including but not limited to products of the tniR resolvase gene, the Cre-Lox recombination system, and the FLP-FRT recombination system. By encoding additional factors alongside the transposon- and CRISPR-associated factors as well as the guide RNA, the modified transposon machinery encoding the Type V-K CRISPR-Cas systems, or any other system configured for replicative transposition, as described herein, regain the autonomous ability to catalyze not only replicative copy-and-paste transposition, but the downstream resolution of the cointegrate product, without homologous recombination. This strategy is depicted in FIG. 5.

In one embodiment, tniR and its accompanying res site—the DNA sequence recognized by the TniR resolvase—are encoded together with the 4 genes for RNA-guided integration within the transposons harboring Type V-K CRISPR-Cas systems (cas12k, tnsB, tnsC, and tniQ); the tniR gene may be encoded within the mini-transposon itself as part of the cargo on the donor DNA, or may be encoded outside of the mini-transposon in trans. However, the res region is included within the mini-transposon, such that the duplicated res sites upon cointegrate product formation can be acted upon by the TniR resolvase, leading to resolution of the cointegrated product, and a final genomic integration product that harbors a single integrated transposon flanked by 5-bp TSD, but no additional exogenous sequence. The tniR and accompanying res sequence may be derived from Tn5053 or from Tn5053-like transposons. The resolvase gene may be encoded off of constitutive or inducible promoters and may be co-delivered together with the transposase and Cas12k/sgRNA machinery, or may be delivered in a subsequent step, such that an initial cointegrate product is acted upon only after the initial transposition has taken place.

A different gene falling within the same resolvase/recombinase family may be used, such as a Tn3 resolvase (also known as TnpR) and its accompanying res sites. In another embodiment, a res site for Tn3 resolvase is included within the genetic payload of the mini-transposon, and the Tn3 resolvase is expressed in trans, together with the 4 genes for RNA-guided integration within the transposons harboring Type V-K CRISPR-Cas systems (cas12k, tnsB, tnsC, and tniQ); the tnpR gene may be encoded within the mini-transposon itself as part of the cargo on the donor DNA, or it may be encoded outside of the mini-transposon. Formation of a cointegrate leads to duplication of the transposon cargo and the Tn3 resolvase res site, and Tn3 resolvase can recognize these duplicated res sites and catalyze their recombination, leading to resolution of the cointegrate and a final genomic integration product that harbors a single integrated transposon flanked by 5-bp TSD, but no additional exogenous sequence. The Tn3 resolvase may be encoded off of constitutive or inducible promoters and may be co-delivered together with the transposase and Cas12k/sgRNA machinery, or may be delivered in a subsequent step, such that an initial cointegrate product is acted upon only after the initial transposition has taken place.

In another embodiment, a single lox site (e.g. loxP) is included within the genetic payload of the mini-transposon, and the Cre recombinase is expressed in trans, together with the 4 genes for RNA-guided integration within the transposons harboring Type V-K CRISPR-Cas systems (cas12k, tnsB, tnsC, and tniQ); the Cre gene may be encoded within the mini-transposon itself as part of the cargo on the donor DNA, or it may be encoded outside of the mini-transposon. Upon cointegrate production formation, the duplicate transposon will have also duplicated the loxP sites as directed repeats, and the Cre recombinase acting upon these tandem loxP sites will lead to a deletion of the intervening sequence, leading to resolution of the cointegrate and a final genomic integration product that harbors a single integrated transposon flanked by 5-bp TSD, but no additional exogenous sequence. Note that this strategy is conceptually similar to the natural resolution that can occur at low frequency by homologous recombination, but because it relies on a sequence-specific recombinase that is provided as part of the RNA-guided integrase technology, it remains an autonomous step not reliant on host homologous recombination factors; allowing for RNA-guided DNA integration in cell types that do not express host DNA repair factors for homologous recombination, but still allow for efficient recombination between the two tandem duplicated transposons within the cointegrate to result in a clean transposition product. The Cre recombinase may be encoded off of constitutive or inducible promoters and may be co-delivered together with the transposase and Cas12k/sgRNA machinery, or may be delivered in a subsequent step, such that an initial cointegrate product is acted upon only after the initial transposition has taken place.

In another embodiment, Cre-Lox is replaced with the FLP-FRT recombination system, or another similar recombination system.

Example vectors encoding the systems described herein are provided in SEQ ID NO: 15-38 and additional vectors appropriate for the methods and uses described herein may be found in International Application No. PCT/US20/21568.

### Example 2

### Resolving Cointegrate Products

Chemically competent E. coli cells were transformed with a plasmid containing Cre recombinase under the control of an arabinose-inducible promoter. Transformed cells were then induced for chemical competency and were transformed with a plasmid containing a Type V-K CRISPR-Cas transposition system (cas12k, tnsB, tnsC, and tniQ), and a plasmid with the respective mini-transposon containing the loxP site. Cells were incubated at 37° C. on selective LB agar for 30 hours to induce RNA-guided transposition. Cells were then scraped and plated on selective LB agar containing 7 mM arabinose for 24 hours to induce for expression of Cre, and genomic DNA was then extracted from the scraped cell population. The experiment was repeated using the TniR resolvase and the TniR res site within the mini-Tn instead of the Cre/loxP system.

Whole-genome sequencing is performed on extracted genomic DNA using the Pacific Biosciences Sequel II long-read sequencing platform. Circular-consensus-sequence reads corresponding to genomic transposon insertion may be analyzed to determined existence of simple insertions or cointegrate formation.

The scope of the present invention is not limited by what has been specifically shown and described hereinabove. Those skilled in the art will recognize that there are suitable alternatives to the depicted examples of materials, configurations, constructions and dimensions. Variations, modifications and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A system for RNA-guided DNA integration comprising:
(a) an engineered Clustered Regularly Interspaced Short Palindromic Repeats (CRISPR)-CRISPR associated (Cas) (CRISPR-Cas) system, and/or one or more vectors encoding the engineered CRISPR-Cas system, wherein the engineered CRISPR-Cas system comprises: (a) at least one Cas protein, and (b) a guide RNA (gRNA);
(b) an engineered transposon system, and/or one or more vectors encoding the engineered transposon system, wherein the engineered transposon system is configured for replicative transposition;
(c) a recombinase, or catalytic domain thereof, and/or one or more vectors encoding a recombinase, or catalytic domain thereof; and
(d) at least one donor nucleic acid to be integrated, wherein the donor nucleic acid comprises a recognition site for the recombinase and a cargo nucleic acid flanked by at least one transposon end sequence.

2-3. (canceled)

4. The system of claim 1, wherein the recombinase, or catalytic domain thereof, comprises a tyrosine recombinase.

5. The system of claim 1, wherein the recombinase comprises Cre recombinase, a mutant, variant, or catalytic domain thereof and the recognition site comprises a lox site or variant thereof.

6-7. (canceled)

8. The system of claim 1, wherein the recombinase comprises flippase (FLP) recombinase, a mutant, variant, or catalytic domain thereof and the recognition site comprises a flippase recognition target (FRT) site or variant thereof.

9-10. (canceled)

11. The system of claim 1, wherein the recombinase, or catalytic domain thereof, comprises a serine recombinase.

12. The system of claim 1, wherein the recombinase comprises TniR resolvase, a mutant, variant, or catalytic domain thereof and the recognition site comprises a resolution (res) sequence or variant thereof.

13-14. (canceled)

15. The system of claim 1, wherein the recombinase comprises a Tn3-like resolvase, mutant, variant, or catalytic domain thereof and the recognition site comprises a resolution (res) sequence or variant thereof.

16-17. (canceled)

18. The system of claim 1, wherein the engineered CRISPR-Cas system comprises a Type V system or a Type I system.

19. The system of claim 1, wherein the engineered CRISPR-Cas system comprises Cas12k.

20. The system of claim 1, wherein the engineered CRISPR-Cas system comprises Cas5, Cas6, Cas7, Cas8, or a combination thereof.

21. The system of claim 1, wherein the engineered transposon system lacks a resolvase.

22. The system of claim 1, wherein the engineered transposon system comprises one or more of TnsB and TnsC, inactive TnsA, and TniQ.

23-24. (canceled)

25. The system of claim 1, wherein the donor nucleic acid comprises a human nucleic acid sequence.

26. The system of claim 1, wherein the cargo nucleic acid comprises the recognition site for the recombinase.

27. The system of claim 1, further comprising a target nucleic acid.

28. (canceled)

29. The system of claim 1, wherein the system is a cell-free system.

30-31. (canceled)

32. A method for resolving cointegration products resulting from RNA-guided nucleic acid integration, wherein the method comprises introducing into a cell a system of claim 1,
wherein the cell comprises a nucleic acid sequence with a target site and the donor nucleic acid is integrated downstream of the target site.

33. The method of claim 32, wherein the recombinase, or catalytic domain thereof, and/or one or more vectors encoding a recombinase, or catalytic domain thereof is introduced to the cell after the introduction of the engineered CRISPR-Cas system, the engineered transposon system, and the at least one donor nucleic acid.

34-64. (canceled)

65. A kit comprising:
(a) an engineered CRISPR-Cas system, and/or one or more vectors encoding the engineered CRISPR-Cas system, wherein the CRISPR-Cas system comprises: (a) at least one Cas protein, and (b) a guide RNA (gRNA);
(b) an engineered transposon system, and/or one or more vectors encoding the engineered transposon system, wherein the engineered transposon system is configured for replicative transposition; and
(c) a recombinase, or catalytic domain thereof, and/or one or more vectors encoding a recombinase, or catalytic domain thereof.

66-67.

68. The kit of claim 65, further comprising at least one donor nucleic acid to be integrated, wherein the donor nucleic acid comprises a recognition site for the recombinase and a cargo nucleic acid flanked by at least one transposon end sequence.


SEQUENCE LISTING (45 sequences)

SEQ ID NO: 1 - protein, 349 aa, Artificial sequence
  Synthetic
PKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 2 - DNA, 34 bp, Artificial sequence
  Synthetic
ataacttcgtatagcatacattatacgaagttat

SEQ ID NO: 3 - protein, 423 aa, Unknown
  Synthetic
MPQFDILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 4 - DNA, 34 bp, Artificial sequence
  Synthetic
gaagttcctattctctagaaagtataggaacttc

SEQ ID NO: 5 - protein, 204 aa, Unknown
  Synthetic
MLIGYMRVSKADGSQATDLQRDALIAAGVDPVHLYEDQASGMREDRPGLTSCLKALRTGDTLVVWKLDRLGRDLRHLINTVHDLTGRGIGLKVLTGHGAAIDTTTAAGKLVFGIFAALAEFERELIAERTIAGLASARARGRKGGRPFKMTAAKLRLAMAAMGQPETKVGDLCQELGVTRQTLYRHVSPKGELRPDGEKLLSRI

SEQ ID NO: 6 - DNA, 60 bp, Artificial sequence
  Synthetic
cggcgagaactttctggctcacactgtcacataatcgaacgtatatgtgacaggtacgac

SEQ ID NO: 7 - protein, 185 aa, Unknown
  Synthetic
MRLFGYARVSTSQQSLDLQVRALKDAGVKANRIFTDKASGSSTDREGLDLLRMKVEEGDVILVKKLDRLGRDTADMIQLIKEFDAQGVAVRFIDDGISTDGDMGQMVVTILSAVAQAERRRILERTNEGRQEAKLKGIKFGRRRTVDRNVVLTLHQKGTGATEIAHQLSIARSTVYKILEDERAS

SEQ ID NO: 8 - DNA, 120 bp, Artificial Sequence
  Synthetic
ccgtacgaaatgttataaattatcggacatcgtaaaactgttacattaatatgtctattaaatcgtaaatttgtaataatagacatgagttgtccgatattcgatttaaggtacattttt

SEQ ID NO: 9 - protein, 381 aa, Artificial sequence
  Synthetic
MGSSHHHHHHSSGLVPRGSHGGGSAAAMGTRLPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 10 - DNA, 1050 bp, Artificial sequence
  Synthetic
ccgaaaaaaaaacgcaaagtgtccaacctgctgacggtgcaccagaatctgccggcactgccggtcgatgcaaccagtgacgaagtgcgcaaaaatctgatggatatgtttcgtgaccgccaagccttcagcgaacatacgtggaaaatgctgctgtcggtttgccgtagctgggcggcctggtgtaaactgaacaatcgcaaatggtttccggcagaaccggaagatgtgcgtgactatctgctgtacctgcaggcacgtggtctggcagttaaaaccatccagcaacatctgggccaactgaacatgctgcaccgtcgctctggtctgccgcgtccgagtgattccaatgccgtcagtctggtgatgcgtcgcattcgtaaagaaaacgtggatgcaggcgaacgcgctaaacaggcactggcttttgaacgtaccgatttcgaccaagttcgctctctgatggaaaacagtgatcgttgccaggacatccgcaatctggcattcctgggtattgcttataacaccctgctgcgcattgcagaaatcgctcgtattcgcgtgaaagatatcagccgtacggacggcggtcgcatgctgattcacatcggccgtaccaaaacgctggtttccaccgcgggcgtcgaaaaagccctgtcactgggtgtcacgaaactggtggaacgctggatttcagtttcgggcgtcgcagatgacccgaacaattacctgttttgtcgtgtgcgcaaaaatggtgttgcagctccgagcgctacctctcagctgagtacgcgtgcgctggaaggcatcttcgaagccacccatcgcctgatttatggcgcgaaagatgacagcggtcagcgttacctggcatggtccggtcactcagctcgtgttggtgcagcacgtgatatggcacgtgcaggtgtctctatcccggaaattatgcaggccggcggttggacgaacgtgaatattgttatgaactatattcgtaacctggactctgaaacgggtgcgatggtgcgtctgctggaagatggcgactga

SEQ ID NO: 11 - DNA, 1146 bp, Artificial sequence
  Synthetic
atgggctcatcgcatcatcatcatcatcattcatcgggtctggttccgcgcggttcacacggtggtggttcggcggccgctatgggcacccgcctgccgaaaaaaaaacgcaaagtgtccaacctgctgacggtgcaccagaatctgccggcactgccggtcgatgcaaccagtgacgaagtgcgcaaaaatctgatggatatgtttcgtgaccgccaagccttcagcgaacatacgtggaaaatgctgctgtcggtttgccgtagctgggcggcctggtgtaaactgaacaatcgcaaatggtttccggcagaaccggaagatgtgcgtgactatctgctgtacctgcaggcacgtggtctggcagttaaaaccatccagcaacatctgggccaactgaacatgctgcaccgtcgctctggtctgccgcgtccgagtgattccaatgccgtcagtctggtgatgcgtcgcattcgtaaagaaaacgtggatgcaggcgaacgcgctaaacaggcactggcttttgaacgtaccgatttcgaccaagttcgctctctgatggaaaacagtgatcgttgccaggacatccgcaatctggcattcctgggtattgcttataacaccctgctgcgcattgcagaaatcgctcgtattcgcgtgaaagatatcagccgtacggacggcggtcgcatgctgattcacatcggccgtaccaaaacgctggtttccaccgcgggcgtcgaaaaagccctgtcactgggtgtcacgaaactggtggaacgctggatttcagtttcgggcgtcgcagatgacccgaacaattacctgttttgtcgtgtgcgcaaaaatggtgttgcagctccgagcgctacctctcagctgagtacgcgtgcgctggaaggcatcttcgaagccacccatcgcctgatttatggcgcgaaagatgacagcggtcagcgttacctggcatggtccggtcactcagctcgtgttggtgcagcacgtgatatggcacgtgcaggtgtctctatcccggaaattatgcaggccggcggttggacgaacgtgaatattgttatgaactatattcgtaacctggactctgaaacgggtgcgatggtgcgtctgctggaagatggcgactga

SEQ ID NO: 12 - DNA, 1272 bp, Artificial sequence
  Synthetic
atgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 13 - DNA, 615 bp, Artificial sequence
  Synthetic
atgctgatcggctacatgcgcgttagcaaagccgatggtagccaagccaccgatctgcagcgtgatgcgctgattgcggcgggcgttgatccggttcatctgtacgaagatcaagccagcggcatgcgcgaagatcgtccgggtctgaccagctgtctgaaagcgctgcgcacgggcgatacgctggtggtttggaaactggatcgtctgggtcgcgatctgcgccatctgatcaataccgtgcatgatctgacgggccgcggcatcggtctgaaagttctgacgggtcatggcgcggccattgataccaccacggccgcgggtaaactggtgttcggcatctttgccgcgctggccgagttcgaacgcgaactgatcgccgaacgcaccattgcgggtctggccagtgcccgtgcccgtggtcgcaaaggtggtcgtccgttcaaaatgacggcggcgaaactgcgtctggcgatggcggcgatgggccaaccggaaaccaaagttggcgatctgtgccaagaactgggcgttacccgtcagacgctgtaccgccacgttagcccaaaaggcgaactgcgcccagatggcgaaaaactgctgagccgcatctag

SEQ ID NO: 14 - DNA, 558 bp, Artificial sequence
  Synthetic
atgcgactttttggttacgctcgggtctcaaccagtcagcagtctcttgatcttcaggtcagagcactcaaagacgcaggtgtgaaagcaaaccgtatatttaccgataaggcatccggcagttcaacagaccgggaagggctggatttgctgaggatgaaggtggaggaaggtgatgtcattctggttaagaagctcgaccgtcttggccgcgacactgccgatatgatccaactgataaaggaatttgacgctcagggcgtggcagtccggttcattgatgacgggatcagtaccgacggtgatatggggcaaatggtggtcaccatcctgtcggctgtggcacaggctgaacgccggaggatcctagaacgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtga

SEQ ID NO: 15 - DNA, 11860 bp, Artificial sequence
  Synthetic
acctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtggagatataccatgggtgaactgccgagtaggtagctgataacgagacctctgtcttgtcagctagggtggtctcgtgaactgccgagtaggtagctgataacggatccgaattcgagcgaaggagatatacatatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatattttctgtctttggttagctgagttccaaagcgatgagtttaaccgttcgttttatgtgtcgaggtggtaaatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaagagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgagctctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactgtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgacctttaataaggagatataccatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgctcaaccgattcgatttttatattatctcaatggtcagtctcactcctatgttcctgactttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggccagttatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcattgaacctgcacggtaccgagctcgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagc

SEQ ID NO: 16 - DNA, 11860 bp, Artificial sequence
  Synthetic
aacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtggagatataccatgggtgaactgccgagtaggtagctgataacgagaatattgtggcaaaagcgcataaggtgctgtgaactgccgagtaggtagctgataacggatccgaattcgagcgaaggagatatacatatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatattttctgtctttggttagctgagttccaaagcgatgagtttaaccgttcgttttatgtgtcgaggtggtaaatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaagagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgagctctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactgtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgacctttaataaggagatataccatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgctcaaccgattcgatttttatattatctcaatggtcagtctcactcctatgttcctgactttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggccagttatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcattgaacctgcacggtaccgagctcgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcaga

SEQ ID NO: 17 - DNA, 11860 bp, Artificial sequence
  Synthetic
aacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtggagatataccatgggtgaactgccgagtaggtagctgataacgagacctctgtcttgtcagctagggtggtctcgtgaactgccgagtaggtagctgataacggatccgaattcgagcgaaggagatatacatatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatattttctgtctttggttagctgagttccaaagcgatgagtttaaccgttcgttttatgtgtcgaggtggtaaatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaagagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgagctctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactgtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgacctttaataaggagatataccatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgctcaaccgattcgatttttatattatctcaatggtcagtctcactcctatgttcctgcctttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggccagttatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcattgaacctgcacggtaccgagctcgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcaga

SEQ ID NO: 18 - DNA, 11860 bp, Artificial sequence
  Synthetic
aacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtggagatataccatgggtgaactgccgagtaggtagctgataacgagaatattgtggcaaaagcgcataaggtgctgtgaactgccgagtaggtagctgataacggatccgaattcgagcgaaggagatatacatatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatattttctgtctttggttagctgagttccaaagcgatgagtttaaccgttcgttttatgtgtcgaggtggtaaatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaagagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgagctctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactgtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgacctttaataaggagatataccatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgctcaaccgattcgatttttatattatctcaatggtcagtctcactcctatgttcctgcctttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggccagttatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcattgaacctgcacggtaccgagctcgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcaga

SEQ ID NO: 19 - DNA, 11862 bp, Artificial sequence
  Synthetic
gcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtggagatataccatgggtgaactgccgagtaggtagctgataacgagacctctgtcttgtcagctagggtggtctcgtgaactgccgagtaggtagctgataacggatccgaattcgagcgaaggagatatacatatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatattttctgtctttggttagctgagttccaaagcgatgagtttaaccgttcgttttatgtgtcgaggtggtaaatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaagagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgagctctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactgtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgacctttaataaggagatataccatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgctcaaccgattcgatttttatattatctcaatggtcagtctcactcctatgttcctgcctttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggccagttatggcgctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcattgaacctgcacggtaccgagctcgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgg

SEQ ID NO: 20 - DNA, 11862 bp, Artificial sequence
  Synthetic
gcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtggagatataccatgggtgaactgccgagtaggtagctgataacgagaatattgtggcaaaagcgcataaggtgctgtgaactgccgagtaggtagctgataacggatccgaattcgagcgaaggagatatacatatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatattttctgtctttggttagctgagttccaaagcgatgagtttaaccgttcgttttatgtgtcgaggtggtaaatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaagagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgagctctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactgtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgacctttaataaggagatataccatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgctcaaccgattcgatttttatattatctcaatggtcagtctcactcctatgttcctgcctttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggccagttatggcgctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcattgaacctgcacggtaccgagctcgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgg

SEQ ID NO: 21 - DNA, 9097 bp, Artificial sequence
  Synthetic
aacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtactaatagcgccgcagttcatgctctttaagagtctctgtactgtggaaaatctgggttagtttgacggttggaaaaccgttttgctttctgaccctggtagctgcccgcttctcatgctctgacttttcacgttatgtggaaaaagtaacgtaatttcgttagttaagacttaccgtaaaaagtcagttctgatgctgctgtcgcaagacaggataggtgcgctcccagcaaaaggagtatgtcttgaaaaagactagccgttctagtaacggtgcggattaccgcagtggtggctactgaatcacccccttcgtcgggggaaccctccaaaaggtgggttgaaagcgagaccctcgacgagggtctcggtaccgttaagtataagaaggagatatacatatgagcacaatcacgattcaatgccgcctagttgcagaagaagcgaccctgcgctacttttgggaacttatggctgagaagaatacgccactcattaatgaattgttggagcagttaggacaacatccagattttgatacttgggtacaagcaggcaagatgcctgaaaagacagttgagaacttgtgcaagtccctagaagatcgagaaccttttgcaaatcagcccggtcgttttcgtacttcggcagttgctctggtaaagtacatttacaaatcctggtttgcgttacaaaaacgtcgggcggacagactagaaggtaaagagcgctggttaaagatgctcaaaagtgatgtggaactggaacgagaaagcaactgtagtttggatattattcgagctaaagctggggaaattcttgctaaagttactgaggggtgcgctccgagcaatcaaacatcctctaagagaaaaaagaagaaaactaaaaaaagccaagcgacaaaagatctccctacgcttttcgagattatattaaaggcttacgaacaagctgaagaaagcctgacacgagctgcccttgcttatctccttaaaaatgactgtgaagtcagtgaagttgatgaagattcagagaaatttaaaaagcgtcggcgtaaaaaggaaatagaaattgaacgtttaagaaatcaactgaaaagtcggatacctaaaggtcgcgatttaaccggagataagtggttgaaaacgctagaagaagccacccgaaatgttcctgaaaatgaggatgaggcaaaggcttggcaagcccaactcttgagagaagctagttctgttccttttcctgtagcttatgaaactagtgaagatatgacttggtttaccaacgagcaaggtcgaatctttgtttactttaatggttcagccaaacataaatttcaagtttattgcgatcgccgtcaacttcattggtttcaacgttttgtagaggactttcaaatcaagaaaaatggtgataaaaaaggtagcgaaaaggagtaccctgctgggttattgacgctttgttccactcgtctgagatggaaagaaagcgcggaaaaaggagatccttggaatgttcatcgcctaattctttcttgcactatcgatacacgcttatggacacttgaaggaacggaacaggtgcgagctgaaaagatcgctcaggttgaaaaaactattagcaagcgggagcaagaagttaatctgagtaagactcaacttgagcgtttacaagccaagcacagtgagagggagagattgaacaacatttttcccaaccgccctagcaagcccagttaccggggtaaatctcatattgcgatcggtgtaagtttcagtttggaaaacccagccactgttgccgtcgtagatgttgcaaccaagaaagtccttacttaccgcagtttcaaacaattactcggagataactacaaccttgcaaaccggctgcggcaacaaaagcaacgtctttctcacgaacgtcacaaagctcagaaacaaggcgcaccaaattcatttggagactcagagttagggcagtatgtagacagattgcttgcaaaatcgattgtcgcgatcgcaaaaacttatcaagctagtagtattgtcttgcctaaacttcgctatatgagggaaatcatacataatgaagttcaagctaaagccgaaaaaaagattccaggatataaagaaggtcaaaaacaatatgccaaacagtatcggataagcgttcatcaatggagttataatagattatcacagattcttgaaagccaagctacaaaagctggaatttctattgaacgtggatcgcaagtaatccaaggtagctcccaagaacaagcacgagatttagctttatttgcctataacgaacgccaacttagtctgggttagtaacctttaataaggagatataccatgggcttagacgaagaatttgagttcactgaggaattgacgcaagctccagatgttattgtgcttgacaagagtcattttgtggtagacccatcccaaattattctgcaaacatcggataagcataaactgagatttaatcttatcaagtggttcgctgaatcccccaacattactattaaatctcagagaaaacaggcagtcgtagatacccttggtgtttctactcggcaggtggagcgtctcctcaagcaatatcataatggtgagttatcagagacagcgggagtacaacgctcagataaggggaaacttcgcatcagccaatactgggaagattatattaaaacaacttatgaaaaaagtctgaaagataaacatccaatgttgccagcagctgtcgtgcgggaagtgaagcgacacgcgatcgttgacctcggactgaaaccaggagattatccccatccagccaccatctatagaaatttagctcctttaatcgagcaacacacgcggaagaaaaaagttagaaatccgggttcgggatcttggctgaccgttgtcacgcgagatggtcagctactgaaggctgactttagtaaccaaattatccaatgcgaccataccgagctggatattcatatagttgatagtcatggcagtttattgtctgaccgtccttggctaactactgttgtcgatacttattcaagttgcattcttggttttcatttatggatcaaacaaccaggttctacggaggtagctcttgccttgagacacgccattctacccaaaaattatccagaggactacaaacttggcaaagtttgggaaatatatggacctccttttcagtatttttttactgatggtggaaaagattttaattcaaagcatctaaaagccattggtaagaaattaggatttcagtgcgaactacgcaatcgcccgccccaaggtggaattgtagaacgtctttttaaaaccattaatactcaagttcttaaagagcttcccggttacacgggggcaaacgttcaagaacgcccgaaaaatgcagagaaagaagcctgtttaacaatacaggatttggataaaattctagccagcttcttttgtgatatttataaccacgaaccctatcccaaggagcctcgcaacacgagatttgaacgctggtttaaaggtatggggggaaaattacccgaacctttggacgagcgagaattggatatttgtttgatgaaagaagcacaacgagttgttcaagctcatggtagcatacagtttgaaaacctaatttatcgaggagaagctctgaaggcatatagaggtgaatacgtcaccctaagatacgatcccgaccatgtcctgactttatatgtctatagttgcgaagctgatgataatgcagaagagttcttgggctatgctcacgctataaacatggatactcatgacttgagtatagaagaattgaaaactctcaataaagaacgaagtaaagctcgtagcgatcattacaactatgatgctttattagcattgggtaaacgcaaagaacttgtagaagaaagaaaacaagacaagaaagcaaaaaggcaatcagagcaaaaacgtctccgcactgcttccaagaaaaactcaaatgttattgaactacgcaaaagtagagcttcgagttcttcaagtaaagatgaccgacaggaaattttaccagagagagtttctagggacgaactaaagccggagaaaacagaactgaaatatgaggaaaatctgttagcacagaccgatactcaaaaacaggagagacataagttagttgtctctgaccgtaaaaaaaatttaaagaacatttggtgataaaggaggtaaataatggccatatctcaactggcaacccaaccatttgtcgaagtcttacctccagagttagatagcaaagctcaaattgctaaaactattgatattgaggagcttttcagaattaattttatcacaaccgatcggtcatcagaatgtttcagatggttagatgagctgcgtatcctgaaacaatgcggtcggatcattggaccaagaaatgtaggcaaaagccgagcagtgcttcactatcgtaatgaggataaaaaacgagtttcttatgtaaaggcttggtcggcatcgagttctaaacgtctattttcacaaatcttaaaggatattaaccatgctgcatcaacaggtaaacgacaggatttacgtccaagattagctggtagtcttgaactatttggattagaattggtcattgttgataatgctgaaaatcttcaaaaagaggcgctcttagatttaaagcaactttttgaagaatgccatgttcctattgtcttagttggaggtaaggagttagatgatattttagaagattttgatttgttgactaactttccaacgttatatgagtttgaacgattggaacatgatgattttataaaaacattaaagacaatcgaattggatattttatctcttccagaagcgtctaagttaagtgagggtaacatatttgcaattttagctgagagtacaggtgggaaaataggaattttagtcaagatactaactaaagcagttttacattctcttaaaaagggatttggtaaagttgatgaaagcattttagaaaaaatcgcaagtcgttatggaacaaaatacgttcccattgaaaacaaaaatagaaacgattaatagaggaggatataaatgattgaagatgatgaaattcgtctaaggttgggctatgttgaaccacacccaggtgaaagcatcagtcattatttgggacggcttcgtcgatttaaggctaacagccttccttcaggatatgctctaggaaaaattgcgggtcttgggtctgttctgactcgatgggaaaagttgtacttcaacccatttcccactcaacaagaactagaggcattagcacaagtgattcaagtggaggttgagaaattaagagagatgctacccactaaaggtgtcacaatgatgcctcgacccattcgattatgtgcggcttgctatgcagagtcaccttatcaccggatcgagtggcagtttaaggacaaaatgaagtgcgatcgccatcagttgcgcttattaacaaaatgcacaaactgtcagacaccattccccattcctgctgattgggaaaaaggagaatgctcgcattgttttttgtcttttgctaagatggtgaagtgtcaaaagcgtcgttagtaacctaggctgctgccaccgctgagcaataactagcataaccccttggggcctctaaacgggtcttgaggggttttttgctgaaacctcaggcatttgagaagcacacggtcacactgcttccgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcaga

SEQ ID NO: 22 - DNA, 9097 bp, Artificial sequence
  Synthetic
aacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacgtactaatagcgccgcagttcatgctctttaagagtctctgtactgtggaaaatctgggttagtttgacggttggaaaaccgttttgctttctgaccctggtagctgcccgcttctcatgctctgacttttcacgttatgtggaaaaagtaacgtaatttcgttagttaagacttaccgtaaaaagtcagttctgatgctgctgtcgcaagacaggataggtgcgctcccagcaaaaggagtatgtcttgaaaaagactagccgttctagtaacggtgcggattaccgcagtggtggctactgaatcacccccttcgtcgggggaaccctccaaaaggtgggttgaaagacggggaatctgctccggcggatgtaccgttaagtataagaaggagatatacatatgagcacaatcacgattcaatgccgcctagttgcagaagaagcgaccctgcgctacttttgggaacttatggctgagaagaatacgccactcattaatgaattgttggagcagttaggacaacatccagattttgatacttgggtacaagcaggcaagatgcctgaaaagacagttgagaacttgtgcaagtccctagaagatcgagaaccttttgcaaatcagcccggtcgttttcgtacttcggcagttgctctggtaaagtacatttacaaatcctggtttgcgttacaaaaacgtcgggcggacagactagaaggtaaagagcgctggttaaagatgctcaaaagtgatgtggaactggaacgagaaagcaactgtagtttggatattattcgagctaaagctggggaaattcttgctaaagttactgaggggtgcgctccgagcaatcaaacatcctctaagagaaaaaagaagaaaactaaaaaaagccaagcgacaaaagatctccctacgcttttcgagattatattaaaggcttacgaacaagctgaagaaagcctgacacgagctgcccttgcttatctccttaaaaatgactgtgaagtcagtgaagttgatgaagattcagagaaatttaaaaagcgtcggcgtaaaaaggaaatagaaattgaacgtttaagaaatcaactgaaaagtcggatacctaaaggtcgcgatttaaccggagataagtggttgaaaacgctagaagaagccacccgaaatgttcctgaaaatgaggatgaggcaaaggcttggcaagcccaactcttgagagaagctagttctgttccttttcctgtagcttatgaaactagtgaagatatgacttggtttaccaacgagcaaggtcgaatctttgtttactttaatggttcagccaaacataaatttcaagtttattgcgatcgccgtcaacttcattggtttcaacgttttgtagaggactttcaaatcaagaaaaatggtgataaaaaaggtagcgaaaaggagtaccctgctgggttattgacgctttgttccactcgtctgagatggaaagaaagcgcggaaaaaggagatccttggaatgttcatcgcctaattctttcttgcactatcgatacacgcttatggacacttgaaggaacggaacaggtgcgagctgaaaagatcgctcaggttgaaaaaactattagcaagcgggagcaagaagttaatctgagtaagactcaacttgagcgtttacaagccaagcacagtgagagggagagattgaacaacatttttcccaaccgccctagcaagcccagttaccggggtaaatctcatattgcgatcggtgtaagtttcagtttggaaaacccagccactgttgccgtcgtagatgttgcaaccaagaaagtccttacttaccgcagtttcaaacaattactcggagataactacaaccttgcaaaccggctgcggcaacaaaagcaacgtctttctcacgaacgtcacaaagctcagaaacaaggcgcaccaaattcatttggagactcagagttagggcagtatgtagacagattgcttgcaaaatcgattgtcgcgatcgcaaaaacttatcaagctagtagtattgtcttgcctaaacttcgctatatgagggaaatcatacataatgaagttcaagctaaagccgaaaaaaagattccaggatataaagaaggtcaaaaacaatatgccaaacagtatcggataagcgttcatcaatggagttataatagattatcacagattcttgaaagccaagctacaaaagctggaatttctattgaacgtggatcgcaagtaatccaaggtagctcccaagaacaagcacgagatttagctttatttgcctataacgaacgccaacttagtctgggttagtaacctttaataaggagatataccatgggcttagacgaagaatttgagttcactgaggaattgacgcaagctccagatgttattgtgcttgacaagagtcattttgtggtagacccatcccaaattattctgcaaacatcggataagcataaactgagatttaatcttatcaagtggttcgctgaatcccccaacattactattaaatctcagagaaaacaggcagtcgtagatacccttggtgtttctactcggcaggtggagcgtctcctcaagcaatatcataatggtgagttatcagagacagcgggagtacaacgctcagataaggggaaacttcgcatcagccaatactgggaagattatattaaaacaacttatgaaaaaagtctgaaagataaacatccaatgttgccagcagctgtcgtgcgggaagtgaagcgacacgcgatcgttgacctcggactgaaaccaggagattatccccatccagccaccatctatagaaatttagctcctttaatcgagcaacacacgcggaagaaaaaagttagaaatccgggttcgggatcttggctgaccgttgtcacgcgagatggtcagctactgaaggctgactttagtaaccaaattatccaatgcgaccataccgagctggatattcatatagttgatagtcatggcagtttattgtctgaccgtccttggctaactactgttgtcgatacttattcaagttgcattcttggttttcatttatggatcaaacaaccaggttctacggaggtagctcttgccttgagacacgccattctacccaaaaattatccagaggactacaaacttggcaaagtttgggaaatatatggacctccttttcagtatttttttactgatggtggaaaagattttaattcaaagcatctaaaagccattggtaagaaattaggatttcagtgcgaactacgcaatcgcccgccccaaggtggaattgtagaacgtctttttaaaaccattaatactcaagttcttaaagagcttcccggttacacgggggcaaacgttcaagaacgcccgaaaaatgcagagaaagaagcctgtttaacaatacaggatttggataaaattctagccagcttcttttgtgatatttataaccacgaaccctatcccaaggagcctcgcaacacgagatttgaacgctggtttaaaggtatggggggaaaattacccgaacctttggacgagcgagaattggatatttgtttgatgaaagaagcacaacgagttgttcaagctcatggtagcatacagtttgaaaacctaatttatcgaggagaagctctgaaggcatatagaggtgaatacgtcaccctaagatacgatcccgaccatgtcctgactttatatgtctatagttgcgaagctgatgataatgcagaagagttcttgggctatgctcacgctataaacatggatactcatgacttgagtatagaagaattgaaaactctcaataaagaacgaagtaaagctcgtagcgatcattacaactatgatgctttattagcattgggtaaacgcaaagaacttgtagaagaaagaaaacaagacaagaaagcaaaaaggcaatcagagcaaaaacgtctccgcactgcttccaagaaaaactcaaatgttattgaactacgcaaaagtagagcttcgagttcttcaagtaaagatgaccgacaggaaattttaccagagagagtttctagggacgaactaaagccggagaaaacagaactgaaatatgaggaaaatctgttagcacagaccgatactcaaaaacaggagagacataagttagttgtctctgaccgtaaaaaaaatttaaagaacatttggtgataaaggaggtaaataatggccatatctcaactggcaacccaaccatttgtcgaagtcttacctccagagttagatagcaaagctcaaattgctaaaactattgatattgaggagcttttcagaattaattttatcacaaccgatcggtcatcagaatgtttcagatggttagatgagctgcgtatcctgaaacaatgcggtcggatcattggaccaagaaatgtaggcaaaagccgagcagtgcttcactatcgtaatgaggataaaaaacgagtttcttatgtaaaggcttggtcggcatcgagttctaaacgtctattttcacaaatcttaaaggatattaaccatgctgcatcaacaggtaaacgacaggatttacgtccaagattagctggtagtcttgaactatttggattagaattggtcattgttgataatgctgaaaatcttcaaaaagaggcgctcttagatttaaagcaactttttgaagaatgccatgttcctattgtcttagttggaggtaaggagttagatgatattttagaagattttgatttgttgactaactttccaacgttatatgagtttgaacgattggaacatgatgattttataaaaacattaaagacaatcgaattggatattttatctcttccagaagcgtctaagttaagtgagggtaacatatttgcaattttagctgagagtacaggtgggaaaataggaattttagtcaagatactaactaaagcagttttacattctcttaaaaagggatttggtaaagttgatgaaagcattttagaaaaaatcgcaagtcgttatggaacaaaatacgttcccattgaaaacaaaaatagaaacgattaatagaggaggatataaatgattgaagatgatgaaattcgtctaaggttgggctatgttgaaccacacccaggtgaaagcatcagtcattatttgggacggcttcgtcgatttaaggctaacagccttccttcaggatatgctctaggaaaaattgcgggtcttgggtctgttctgactcgatgggaaaagttgtacttcaacccatttcccactcaacaagaactagaggcattagcacaagtgattcaagtggaggttgagaaattaagagagatgctacccactaaaggtgtcacaatgatgcctcgacccattcgattatgtgcggcttgctatgcagagtcaccttatcaccggatcgagtggcagtttaaggacaaaatgaagtgcgatcgccatcagttgcgcttattaacaaaatgcacaaactgtcagacaccattccccattcctgctgattgggaaaaaggagaatgctcgcattgttttttgtcttttgctaagatggtgaagtgtcaaaagcgtcgttagtaacctaggctgctgccaccgctgagcaataactagcataaccccttggggcctctaaacgggtcttgaggggttttttgctgaaacctcaggcatttgagaagcacacggtcacactgcttccgaattcactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcaga

SEQ ID NO: 23 - DNA, 8853 bp, Artificial sequence
  Synthetic
aacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagcgagaccctcgacgagggtctcggtgaatttttttgttcagaacgctcggtcttgcacaccgggcgttttttctttgtgagtccagaattcactggccactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcaga

SEQ ID NO: 24 - DNA, 8853 bp, Artificial sequence
  Synthetic
aacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaatggataaccgtattaccgcctttgagtgagctgataccgctcgccgcagccgaacgaccgagcgcagcgagtcagtgagcgaggacacaagctttgacagctagctcagtcctaggtataatactagcgtcgacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagacggggaatctgctccggcggatgtgaatttttttgttcagaacgctcggtcttgcacaccgggcgttttttctttgtgagtccagaattcactggccactggccgtcgttttacaacgtcgtgactgggaaaaccctggcgttacccaacttaatcgccttgctatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcaga

SEQ ID NO: 25 - DNA, 4291 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccaagctttgttgatacaaccataaaatgataattacacccataaattgataattatcacacccataaattgatattgcctcttcatggtctaaacttcagtaagtttacgacattttcatgaagaggtcatttccggggatccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttactgcagtagttttgctgaaatactcgattcacaaaaatatcaacttatggttgttttgtgagatatcaatatatggttgttttgtggttaagttgctgattataaataattattaaatatcactttatggttgcatcaacaggtaccgggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 26 - DNA, 4325 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccaagctttgttgatacaaccataaaatgataattacacccataaattgataattatcacacccataaattgatattgcctcttcatggtctaaacttcagtaagtttacgacattttcatgaagaggtcatttccggggatccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatataacttcgtatagcatacattatacgaagttatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttactgcagtagttttgctgaaatactcgattcacaaaaatatcaacttatggttgttttgtgagatatcaatatatggttgttttgtggttaagttgctgattataaataattattaaatatcactttatggttgcatcaacaggtaccgggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 27 - DNA, 4759 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttctgcagcaaagactacctacttaaatgaaccgcaaatgtacattcgcaaattaaatgtcgtaattcgcaaatttgtgtcgtttttcgcaaattaatgtcgtttagaatagtttgtctcatcaattcaattataggaacttttcgcaaattaatgtcgtcctgtttctccatttagtgtcgattaacaaattaatgtcgctgttaacgaattaatgtcgtcgaattagttccaactaacgttaacaaattaatgtcgtctaacataatgtcttgctaggatccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttctagaaaaaccactgattaacactttaggtactaaagtaccactaatgtggcaaaaatgcgacatctaatttgcgaaacaggcaaatcttaataaacgacatttaatttgcgaaaataggatttgcgacatctaatttgcgaaacaggcaaattactcagttttatggataaatagcttgtaagtcctacgcaataaagatctcagctattagaagtaattgcgacactaatttgcgaattgcgacatataatttgcgaatgtacaccaaccccaagcattggtaccgggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 28 - DNA, 4793 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttctgcagcaaagactacctacttaaatgaaccgcaaatgtacattcgcaaattaaatgtcgtaattcgcaaatttgtgtcgtttttcgcaaattaatgtcgtttagaatagtttgtctcatcaattcaattataggaacttttcgcaaattaatgtcgtcctgtttctccatttagtgtcgattaacaaattaatgtcgctgttaacgaattaatgtcgtcgaattagttccaactaacgttaacaaattaatgtcgtctaacataatgtcttgctaggatccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatataacttcgtatagcatacattatacgaagttatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttctagaaaaaccactgattaacactttaggtactaaagtaccactaatgtggcaaaaatgcgacatctaatttgcgaaacaggcaaatcttaataaacgacatttaatttgcgaaaataggatttgcgacatctaatttgcgaaacaggcaaattactcagttttatggataaatagcttgtaagtcctacgcaataaagatctcagctattagaagtaattgcgacactaatttgcgaattgcgacatataatttgcgaatgtacaccaaccccaagcattggtaccgggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 29 - DNA, 4815 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttctgcagcaaagactacctacttaaatgaaccgcaaatgtacattcgcaaattaaatgtcgtaattcgcaaatttgtgtcgtttttcgcaaattaatgtcgtttagaatagtttgtctcatcaattcaattataggaacttttcgcaaattaatgtcgtcctgtttctccatttagtgtcgattaacaaattaatgtcgctgttaacgaattaatgtcgtcgaattagttccaactaacgttaacaaattaatgtcgtctaacataatgtcttgctaggatccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatcggcgagaactttctggctcacactgtcacataatcgaacgtatatgtgacaggtacgacagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttctagaaaaaccactgattaacactttaggtactaaagtaccactaatgtggcaaaaatgcgacatctaatttgcgaaacaggcaaatcttaataaacgacatttaatttgcgaaaataggatttgcgacatctaatttgcgaaacaggcaaattactcagttttatggataaatagcttgtaagtcctacgcaataaagatctcagctattagaagtaattgcgacactaatttgcgaattgcgacatataatttgcgaatgtacaccaaccccaagcattggtaccgggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 30 - DNA, 4665 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttcctaggcatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagactctagaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatttacctcctgcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 31 - DNA, 4699 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttcctaggcatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagactctagaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggataacttcgtataatgtatgctatacgaagttatatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatttacctcctgcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 32 - DNA, 4721 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttcctaggcatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagactctagaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactgtcgtacctgtcacatatacgttcgattatgtgacagtgtgagccagaaagttctcgccgatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatttacctcctgcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 33 - DNA, 4781 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttcctaggcatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagactctagaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccgtacgaaatgttataaattatcggacatcgtaaaactgttacattaatatgtctattaaatcgtaaatttgtaataatagacatgagttgtccgatattcgatttaaggtacatttttatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatttacctcctgcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 34 - DNA, 4695 bp, Artificial sequence
  Synthetic
ccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttcaagcgaaccggaattgccagctggggcgccctctggtaaggttgggaagccctgcaaagtaaactggatggctttcttgccgccaaggatctgatggcgcaggggatcatcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaatgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttcctaggcatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagactctagaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactgaagttcctattctctagaaagtataggaacttcatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatttacctcctgcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattg

SEQ ID NO: 35 - DNA, 6743 bp, Artificial sequence
  Synthetic
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacacatttccccgaaaagtgccacctgcatcgatttattatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattctagaaataattttgtttaactttaagaaggagatataccatgggctcatcgcatcatcatcatcatcattcatcgggtctggttccgcgcggttcacacggtggtggttcggcggccgctatgggcacccgcctgccgaaaaaaaaacgcaaagtgtccaacctgctgacggtgcaccagaatctgccggcactgccggtcgatgcaaccagtgacgaagtgcgcaaaaatctgatggatatgtttcgtgaccgccaagccttcagcgaacatacgtggaaaatgctgctgtcggtttgccgtagctgggcggcctggtgtaaactgaacaatcgcaaatggtttccggcagaaccggaagatgtgcgtgactatctgctgtacctgcaggcacgtggtctggcagttaaaaccatccagcaacatctgggccaactgaacatgctgcaccgtcgctctggtctgccgcgtccgagtgattccaatgccgtcagtctggtgatgcgtcgcattcgtaaagaaaacgtggatgcaggcgaacgcgctaaacaggcactggcttttgaacgtaccgatttcgaccaagttcgctctctgatggaaaacagtgatcgttgccaggacatccgcaatctggcattcctgggtattgcttataacaccctgctgcgcattgcagaaatcgctcgtattcgcgtgaaagatatcagccgtacggacggcggtcgcatgctgattcacatcggccgtaccaaaacgctggtttccaccgcgggcgtcgaaaaagccctgtcactgggtgtcacgaaactggtggaacgctggatttcagtttcgggcgtcgcagatgacccgaacaattacctgttttgtcgtgtgcgcaaaaatggtgttgcagctccgagcgctacctctcagctgagtacgcgtgcgctggaaggcatcttcgaagccacccatcgcctgatttatggcgcgaaagatgacagcggtcagcgttacctggcatggtccggtcactcagctcgtgttggtgcagcacgtgatatggcacgtgcaggtgtctctatcccggaaattatgcaggccggcggttggacgaacgtgaatattgttatgaactatattcgtaacctggactctgaaacgggtgcgatggtgcgtctgctggaagatggcgactgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 36 - DNA, 6159 bp, Artificial sequence
  Synthetic
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacacatttccccgaaaagtgccacctgcatcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattctagaaataattttgtttaactttaagaaggagatataccatgctgatcggctacatgcgcgttagcaaagccgatggtagccaagccaccgatctgcagcgtgatgcgctgattgcggcgggcgttgatccggttcatctgtacgaagatcaagccagcggcatgcgcgaagatcgtccgggtctgaccagctgtctgaaagcgctgcgcacgggcgatacgctggtggtttggaaactggatcgtctgggtcgcgatctgcgccatctgatcaataccgtgcatgatctgacgggccgcggcatcggtctgaaagttctgacgggtcatggcgcggccattgataccaccacggccgcgggtaaactggtgttcggcatctttgccgcgctggccgagttcgaacgcgaactgatcgccgaacgcaccattgcgggtctggccagtgcccgtgcccgtggtcgcaaaggtggtcgtccgttcaaaatgacggcggcgaaactgcgtctggcgatggcggcgatgggccaaccggaaaccaaagttggcgatctgtgccaagaactgggcgttacccgtcagacgctgtaccgccacgttagcccaaaaggcgaactgcgcccagatggcgaaaaactgctgagccgcatctagctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 37 - DNA, 6102 bp, Artificial sequence
  Synthetic
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacacatttccccgaaaagtgccacctgcatcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattctagaaataattttgtttaactttaagaaggagatataccatgcgactttttggttacgctcgggtctcaaccagtcagcagtctcttgatcttcaggtcagagcactcaaagacgcaggtgtgaaagcaaaccgtatatttaccgataaggcatccggcagttcaacagaccgggaagggctggatttgctgaggatgaaggtggaggaaggtgatgtcattctggttaagaagctcgaccgtcttggccgcgacactgccgatatgatccaactgataaaggaatttgacgctcagggcgtggcagtccggttcattgatgacgggatcagtaccgacggtgatatggggcaaatggtggtcaccatcctgtcggctgtggcacaggctgaacgccggaggatcctagaacgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtgactgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 38 - DNA, 6816 bp, Artificial sequence
  Synthetic
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacacatttccccgaaaagtgccacctgcatcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattctagaaataattttgtttaactttaagaaggagatataccatgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataactgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 39 - DNA, 4 bp, Artificial sequence
  Synthetic
tttt

SEQ ID NO: 40 - DNA, 7 bp, Artificial sequence
  Synthetic
  misc_feature (1)..(2): n is a, c, g, or t
nnagaaw

SEQ ID NO: 41 - DNA, 8 bp, Artificial sequence
  Synthetic
  misc_feature (1)..(4): n is a, c, g, or t
nnnngatt

SEQ ID NO: 42 - DNA, 6 bp, Artificial sequence
  Synthetic
  misc_feature (1)..(2): n is a, c, g, or t
nnagaa

SEQ ID NO: 43 - DNA, 6 bp, Artificial sequence
  Synthetic
  misc_feature (1)..(1): n is a, c, g, or t
naaaac

SEQ ID NO: 44 - DNA, 5 bp, Artificial sequence
  Synthetic
gcggg

SEQ ID NO: 45 - DNA, 5 bp, Artificial sequence
  Synthetic
cgccc